(19) United States
(12) Patent Application Publication
(10) Pub. No.: US 2014/0216136 A1
(43) Pub. Date: Aug. 7, 2014

(54) SMART PHONE SENSOR PLATFORM

(71) Applicant: Charles Michael Yim, San Mateo, CA (US)

(72) Inventor: Charles Michael Yim, San Mateo, CA (US)

(73) Assignee: Breathometer, Inc., Burlingame, CA (US)

(21) Appl. No.: 13/927,321

(22) Filed: Jun. 26, 2013

Related U.S. Application Data
(60) Provisional application No. 61/808,933, filed on Apr. 5, 2013.
(60) Provisional application No. 61/665,152, filed on Jun. 27, 2012.

(51) Int. Cl. G01N 33/00; G01N 27/00

(52) U.S. Cl. CPC G01N 33/0004; G01N 27/00
USPC 73/31.05

(57) ABSTRACT

A sensor device containing a gas sensor is exposed to a gas. The gas sensor emits a voltage based on a property of the gas. The voltage output by the gas sensor is received by an integrated circuit (“IC”). The IC may convert the voltage to a digital representation of the voltage. The IC may determine a property of the gas based on the voltage. The digital representation of the voltage or a representation of the property of the gas is then transmitted as data via an audio jack of the sensor device. A computer device receives the data via an audio jack. Using one or more processors, the computer device analyzes the data and generates a recommendation. The recommendation and the property of the gas may be displayed.

### CLAIM OF PRIORITY UNDER 35 U.S.C. §119(e)

The present application claims priority under 35 U.S.C. §119(e) to and incorporates by reference U.S. Provisional Application No. 61/665,152, filed Jun. 27, 2012, entitled “Mobile Phone Breathalyzer.” The present application claims priority to and incorporates by reference U.S. Provisional Application No. 61/808,933, filed Apr. 5, 2013, entitled “Smart Phone Sensor Platform.”

### TECHNICAL FIELD

The subject matter disclosed herein generally relates to the analysis of gas. Specifically, the present disclosure addresses systems and methods to detect substances in air and display the results of the detection.

### BACKGROUND

Breathalyzer devices analyze an exhalation of an individual to determine the amount of alcohol present in the exhalation using a fuel-cell semiconductor sensor. Typically, a fixed conversion ratio is used (e.g., 2100:1) to determine the individual's blood-alcohol content from the individual's breath-alcohol content. Existing devices are expensive, monolithic, and cumbersome.

### BRIEF DESCRIPTION OF THE DRAWINGS

Some embodiments are illustrated by way of example and not limitation in the figures of the accompanying drawings.

FIG. 1 is a network diagram illustrating a network environment suitable for gathering and processing gas data, according to some example embodiments.

FIG. 2 is a block diagram illustrating components of a user device suitable for analyzing a gas, according to some example embodiments.

FIG. 3 is a block diagram illustrating components of a server machine suitable for processing gas data, according to some example embodiments.

FIG. 4 is a graph illustrating a relationship between voltage and blood-alcohol content (“BAC”), according to some example embodiments.

FIG. 5 is a diagram illustrating a device capable of gathering gas data, according to some example embodiments.

FIG. 6-9 are flowcharts illustrating operations of a device in performing a method of analyzing a gas, according to some example embodiments.

FIG. 10 is a block diagram illustrating components of a machine, according to some example embodiments, able to read instructions from a machine-readable medium and perform any one or more of the methodologies discussed herein.

FIG. 11 is a block diagram illustrating communications between two devices performing a method of analyzing a gas, according to some example embodiments.

FIG. 12-22 are block diagrams illustrating screens of user interfaces presented in methods of analyzing a gas, according to some example embodiments.

### DETAILED DESCRIPTION

Example methods and systems are directed to analysis of one or more gases. Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments, it will be evident to one skilled in the art, however, that the present subject matter may he practiced without these specific details.

A device containing a gas sensor is exposed to a gas. The gas sensor emits a voltage based on a property of the gas. For example, the output voltage of the gas sensor may vary based on an electrical property of the gas (e.g., its resistance, capacitance, inductance, or any suitable combination thereof). As another example, the output voltage of the gas sensor may vary based on an optical property of the gas (e.g., its luminosity, visual opacity, transparency to different wavelengths, or any suitable combination thereof). As another example, the output voltage of the gas sensor may vary based on a chemical property of the gas (e.g., its carbon content, its ethanol content, its energy content, its ability to react with different chemical compounds, or any suitable combination thereof).

The voltage output by the gas sensor is received by an integrated circuit (“IC”). In some example embodiments, the IC converts the voltage to a digital representation of the voltage. In some example embodiments, the IC determines a property of the gas based on the voltage. The digital representation of the voltage or a representation of the property of the gas is then transmitted as data via an audio jack.

A device receives the data via an audio jack and, using one or more processors, determines the property of the gas. Based on the property of the gas and, potentially, other information, the one or more processors may determine a derivative property. For example, the concentration of ethanol in the gas, the concentration of a toxic substance in the gas, the concentration of a pathogen in the gas, or any suitable combination thereof. This derivative property may itself be used to determine a further derivative property. For example, the concentration of ethanol in the gas may be used to determine the blood-alcohol content of the person who exhaled the gas. In another example, the concentration of the toxic substance in the gas may be used to determine the toxicity or safety of the gas. In another example, the concentration of the pathogen in the gas may be used to determine the safety of the gas or the efficacy of a quarantine area near to the location from which the sample was taken. As used herein, the term “direct property of the gas” refers to a property of the gas directly measured by the gas sensor, while the term “derivative property of the gas” refers to any property determined based on the direct property of the gas. The general term “property of the gas” includes both direct properties and derivative properties.

Based on the property of the gas, the device may generate a recommendation. The device may then display the property, the recommendation, or both. In some example embodiments, the device may then implement the recommendation, enable the user to implement the recommendation, or bath. For example, if the property of the gas is the BAC of a user, the recommendation may be to call a taxi. In this example, the device may provide a phone number to the user, provide a selection of numbers to call, automatically call a taxi, or any suitable combination thereof.

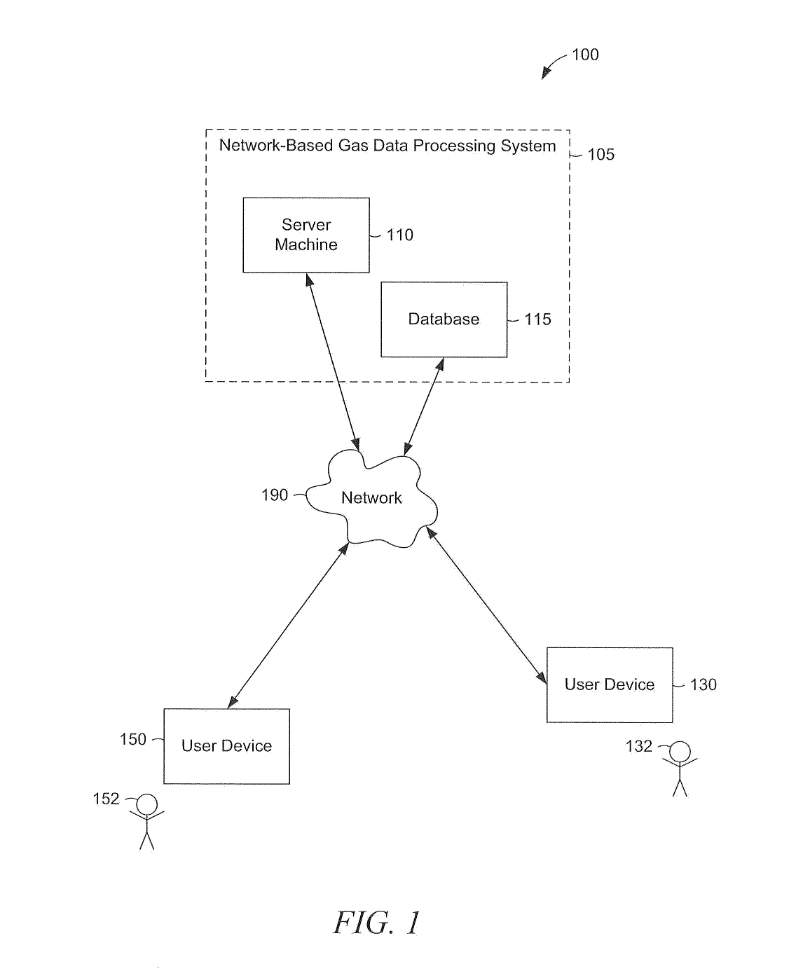

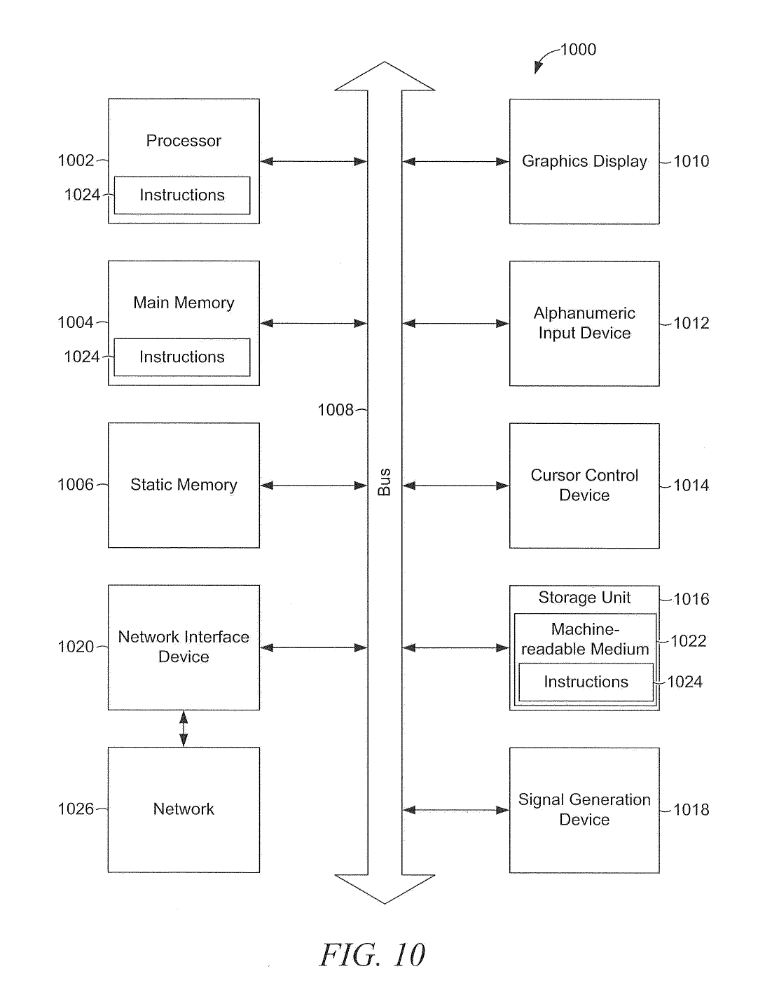

FIG. 1 is a network diagram illustrating a network environment 100 suitable for analyzing a gas, according to some example embodiments. The network environment 100 includes a network-based gas data processing system 105, a server machine 110, a database 115, and devices 130 and 150, all communicatively coupled to each other via a network 190. The server machine 110 and the devices 130 and 150 may each be implemented in a computer system, in whole or in part, as described below with respect to FIG. 10,

The network-based gas data processing system 105 may include the server machine 110 and the database 115. The network-based gas data processing system 105 may provide the elements and services of each of the server machine 110 and the database 115, as described in more detail below.

The server machine 110 may receive data from the user devices 130 and 150. The data may indicate a property of a gas (e.g., the purity of the gas, the density of pollutants in the gas, the number of microorganisms in the gas, or any suitable combination thereof). As an alternative, the data may indicate a derivative property of the gas (e.g., that an individual exhaling the gas has a certain BAC, that a machine emitting the gas is or is not functioning properly, that a quarantine area near the location from which the gas was taken is or is not properly sealed, or any suitable combination thereof). Alternatively, the data may indicate a measurement taken by a sensor (e.g., a voltage measurement, a resistance measurement, a temperature measurement, alight flux measurement, or any suitable combination thereof). In some example embodiments, the measurement is used by the server machine 110 to determine the property of the gas. In some example embodiments, the property of the gas is used by the server machine 110 to determine a property associated with the gas. The server machine 110 may store the data in the database 115. The server machine 110 may send a response to the device 130 or 150 in response to receiving the data. In some example embodiments, the server machine 110 sends a recommendation to the device based on the data received. For example, the server machine 110 may send a recommendation that the user 132 call a cab rather than driving if the data indicates that the user 132 may have an unsafe BAC.

The database 115 may store data and stored procedures that operate on the data. In some example embodiments, the database 115 stores data generated by the user devices 130 and 150. For example, the database 115 may store entries including data representing sensor voltages, time stamps, locations, user identifiers (IDs), properties of gases, user data (e.g., age, weight, height, gender, medical history, geolocation, or any suitable combination thereof), device data (e.g., make, model, global positioning system location, device ID, or any suitable combination thereof) or any suitable combination thereof. The database 115 may also store data tables useful for converting sensor voltages into more meaningful values. For example, the database 115 may store a data table that relates sensor voltages to ethanol concentrations. In an example embodiment, the database 115 stores a lookup table that provides the BAC of an individual based on a sensor voltage, a representation of a sensor voltage, a temperature, a concentration of ethanol, a humidity, and personal information of the individual.

Also shown in FIG. 1 are users 132 and 152. One or both of the users 132 and 152 may be a human user (e.g., a human being), a machine user (e.g., a computer configured by a software program to interact with the device 130), or any suitable combination thereof (e.g., a human assisted by a machine or a machine supervised by a human). The user 132 is not part of the network environment 100, but is associated with the device 130 and may be a user of the device 130. For example, the device 130 may be a desktop computer, a vehicle computer, a tablet computer, a navigational device, a portable media device, or a smart phone belonging to the user 132. Likewise, the user 152 is not part of the network environment 100, but is associated with the device 150. As an example, the device 150 may be a desktop computer, a vehicle computer, a tablet computer, a navigational device, a portable media device, or a smart phone belonging to the user 152.

The user devices 130 and 150 may include a sensor module connected to the computing device via an audio jack. The user devices 130 and 150 may use a communications protocol to send data from the Sensor module to the computing device, from the computing device to the sensor module, or both. In some example embodiments, the user 130 may exhale onto or into the sensor module. In other example embodiments, the user 130 may place the sensor module in a location at which measurements are to be taken.

Any of the machines, databases, or devices shown in FIG. 1 may be implemented in a general-purpose computer modified (e.g., configured or programmed) by software to be a special-purpose computer to perform the functions described herein for that machine, database, or device. For example, a computer system able to implement any one or more of the methodologies described herein is discussed below with respect to FIG. 10. As used herein, a “database” is a data storage resource and may store data structured as a text file, a table, a spreadsheet, a relational database (e.g., an object relational database), a triple store, a hierarchical data store, or any suitable combination thereof. Moreover any two or more of the machines, databases, or devices illustrated in FIG. 1 may be combined into a single machine, and the functions described herein for any single machine, database, or device may be subdivided among multiple machines, databases, or devices.

The network 190 may be any network that enables communication between or among machines, databases, and devices (e.g., the server machine 110 and the device 130). Accordingly, the network 190 may be a wired network, a wireless network (e.g., a mobile or cellular network), or any suitable combination thereof. The network 190 may include one or more portions that constitute a private network, a public network (e.g., the Internet), or any suitable combination thereof.

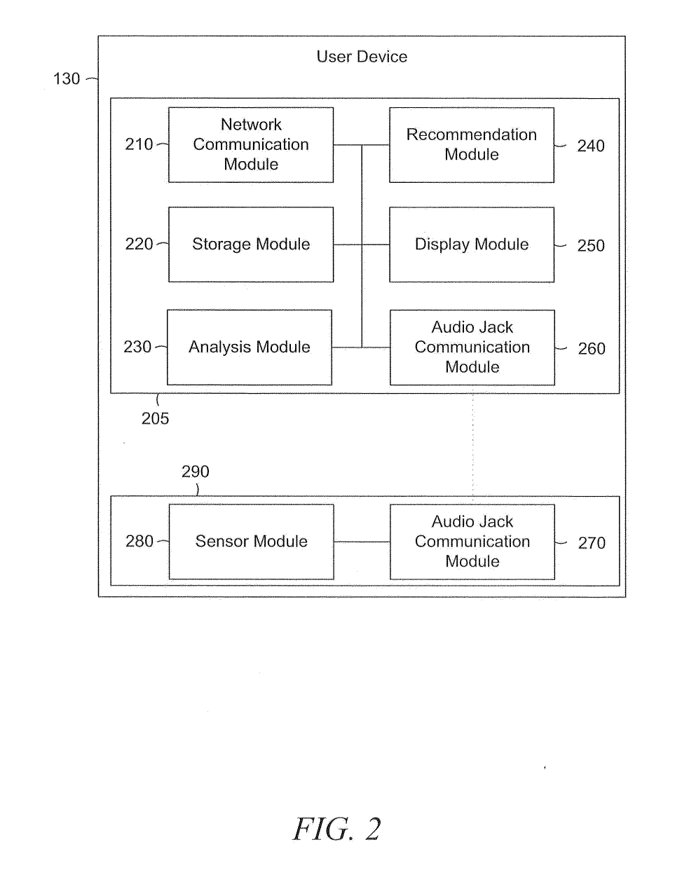

FIG. 2 is a block diagram illustrating components of the user device 130 or 150, according to some example embodiments. The user device 130 is shown as including a computing device 205 and a sensor device 290. The computing device 205 is shown as further including a network communication module 210, a storage module 220, an analysis module 230, a recommendation module 240, a display module 250, and an audio jack communication module 260, all configured to communicate with each other (e.g., via a bus, shared memory, or a switch). The sensor device 290 is shown as further including an audio jack communication module 270 and a sensor module 280, each configured to communicate with the other (e.g., via a bus, shared memory, or a switch). Any one or more of the modules described herein may be implemented using hardware (e.g., a processor of a machine) or a combination of hardware and software. For example, any module described herein may configure a processor to perform the operations described herein for that module. Moreover, any two or more of these modules may be combined into a single module, and the functions described herein for a single module may be subdivided among multiple modules. Furthermore, according to various example embodiments, modules described herein as being implemented within a single machine, database, or device may be distributed across multiple machines, databases, or devices.

The network communication module 210 may send data and commands from the user device 130 to the server machine 110. The communication module 210 may receive data and commands from the server machine 110. In some example embodiments, the communication module 210 sends and receives data over the network 190. In some example embodiments, the communication module 210 sends data received from the sensor module 280 to the server machine 110. The communication module 210 may send data received from the server machine 110 to the display module 250.

The storage module 220 may store and retrieve data. In some example embodiments, the storage module 220 stores a history of sensor values generated by the sensor module 280, a history of BAC levels generated by the analysis module 230, user data, device data, or any suitable combination thereof.

The analysis module 230 may analyze data retrieved by the audio jack communication module 260. In some example embodiments, the audio jack communication module 260 reports a sensor voltage to the analysis module 230. In these example embodiments, the analysis module 230 may convert the sensor voltage value to a value of greater interest to the user. For example, if the sensor operates by reporting a varying voltage based on the resistance of a gas to which the sensor is exposed, this value may be of great interest to an engineer seeking to develop an insulating or conductive gas, but is of little interest to an individual seeking to determine if driving is a legal option. For such an individual, the analysis module 230 may convert the sensor voltage value to a BAC value, to an ethanol concentration value, or both. Similarly, for a user interested in determining if the air in a home has unsafe levels of carbon monoxide, the analysis module 230 nay convert the sensor voltage value to a safety value, to a carbon monoxide concentration value, or both. In other example embodiments, the value received by the analysis module 230 is the value of interest to the user and no conversion by the analysis module 230 is required. In sonic example embodiments, the analysis module 230 sends the resulting values to the network communication module 210, the display module 250, or both. The analysis module 230 may store the resulting values locally for future use. The resulting values may be stored on the server machine 110 after transmission by the network module 210, in some example embodiments, the analysis module 230 determines that the results reported by the audio jack communication module 260 are likely incorrect. In such example embodiments, the analysis module 230 directs the audio jack communication module 260 to request the sensor module 280 to generate new sensor readings. Such a request may be made via the audio jack communication module 270.

The recommendation module 240 may generate a recommendation for the user based on data generated by the sensor module 280, results generated by the analysis module 230, data received by the network communication module 210, data stored in the storage module 220, or any combination thereof. For example, the recommendation module 240 may receive an indication from the analysis module 230 that the user 132 has a BAC over the legal limit. In response, the recommendation module 240 may generate a recommendation that the user 132 take a cab instead of driving a car. In some example embodiments, the recommendation module 240 accesses data from the storage module 220 regarding local taxi cab companies. In other example embodiments, the recommendation module 240 receives a recommendation from the server machine 110 via the network communication module 210. In some example embodiments, the recommendation module 240 provides the recommendation to the display module 250 for display to the user.

The display module 250 may display information to the user or cause such information to be displayed. For example, the display module 250 may display a prompt to the user to blow into or onto the sensor. After the sensor module 280 has reported a sensor value, the display module 250 may display the sensor value. In some embodiments, the sensor module 280 displays a more-relevant value generated by analysis module 230. For example, the analysis module 250 may determine the user's BAC based on the sensor value. Then the analysis module 250 may report the user's BAC to the display module 250. The display module 250 may then display the user's BAC to the user. In some example embodiments, the display module 250 may display a recommendation generated by the recommendation module 240, the server machine 110, or both.

The audio jack communication module 260 may communicate with the audio jack communication module 270 via an audio jack. The audio jack communication module 260 may provide data it receives to network communication module 210 for transmission to server machine 110, to storage module 220 for storage, to the analysis module 230 for analysis, to the recommendation module 240 to be used as the basis for a recommendation, to the display module 250 for display, or any suitable combination thereof. The audio jack communication module 260 may transmit commands to the audio jack communication module 270 via the audio jack. For example, the audio jack communication module 260 may request a sensor reading, request the sensor to be powered up, request the sensor to be powered down, request the audio jack communication module 270 to resend a data packet, request the audio jack communication module 270 to test the communications link, or any suitable combination thereof.

The audio jack communication modules 260 and 270 may communicate via a telephone modem protocol, such as the CCITT v. series of standards. The audio jack communication modules 260 and 270 may communicate via a duration modulation protocol. For example, pulses may be sent at regular intervals, with the duration of the pulse indicating if the bit corresponding to the pulse is a 0 or a 1. In some example embodiments, a short pulse represents a 0 and a long pulse represents a 1. In sonic example embodiments, a short pulse has a duration of 0.01 s and a long pulse has a duration of 0.02 s. The audio jack communication modules 260 and 270 may communicate via a frequency modulation protocol. For example, a 0 bit may be transmitted by transmitting a sound on the audio jack at a particular frequency and holding it for a particular period of time. In this example, a 1 bit may be transmitted by transmitting a sound on the audio jack at a different frequency and holding it for the same period of time. In some example embodiments, the nominal frequency for a 0 bit, denoted as f₀, is 2.2 kHz. In some example embodiments, the nominal frequency for a 1 bit, denoted as is 1.2 kHz. In some example embodiments, f₀ falls into the range 2.083-2.5 kHz. In some example embodiments, f₁ falls into the range 1.042-1.316 kHz. The nominal duration of a 0 bit, denoted t₀, may be 3.3 ms. The nominal duration of a 1 bit, denoted t₁, may be 3.3 ms. In some example embodiments, t₀ is in the range 2.77-3.6 ms. In some example embodiments, t₁ is in the range'2.96-3.8 ms. In some example embodiments, the least significant bit (LSB) is sent first. In some example embodiments, a start hit is sent before the data bits. A stop bit may be sent after the data bits. In some example embodiments, the start bit is a 0 and the stop bit is a 1. A period of silence, denoted t_{silence}, may be used between data segments. In some example embodiments, t,_{silence} is nominally 10.8 ms.

In some example embodiments, the audio jack communication modules 260 and 270 use the protocol shown below.

Downstream (from the device 205 to the device 290)

| | Bit | | | Time- |
|---|---|---|---|---|
| Command | 0-7 | Remarks | Response | out |
| Connect | 0xA5 | Check if device is | Ack | 2 s |
| | | connected | | |
| Warm-up | 0xA7 | Device will start to | Ack | 12 s |
| | | warm up | | |
| Resend | 0xA3 | Resend last command | Last Data | 2 s |
| | | sent | | |
| Measure | 0xAc | After warm-up, send this | Ack | 2 s |
| | | command to measure | | |
| | | again | | |
| CheckLowBattery | 0xA2 | Check if low battery | Voltage | 2 s |
| Calibrate0 | 0xA4 | Calibrate point 0 | Ack | 2 s |
| Calibrate1 | 0xA6 | Calibrate point 1 | Ack | 2 s |
| LED | 0xBx | B0: LED 0 status | Ack | 2 s |
| | | B1: LED 1 status | | |
| | | B2: LED 0 setting, 0 = | | |
| | | normal, 1 = flash | | |
| | | B3: LED 1 setting, 0 = | | |
| | | normal, 1 = flash | | |

Upstream (from the device 290 to the device 205)

| Command | 7 | 6 | 5 | 4 | 3 | 2 | 1 | 0 | Value | Remarks |
|---|---|---|---|---|---|---|---|---|---|---|
| Ready | 1 | 0 | 0 | 0 | 0 | 0 | 0 | 1 | 0x81 | Device |
| | | | | | | | | | | is ready to measure |
| Countdown | 1 | 0 | 0 | 0 | 0 | 0 | 1 | 0 | 0x82 | Device |
| | | | | | | | | | | detected a peak |
| | | | | | | | | | | and start to |
| | | | | | | | | | | countdown |
| Time-out | 1 | 0 | 0 | 0 | 0 | 0 | 1 | 1 | 0x83 | No peak is |
| | | | | | | | | | | detected |
| Ack* | 1 | 0 | 0 | 1 | 1 | Version | | | 0x98-0x9F | Acknowledge |
| Voltage | 1 | 0 | 1 | Voltage | | | | | 0xA0-0xBF | 0xA0: <= 1.1 V, |
| | | | | | | | | | | 0x1F: >= 1.4 V |
| Data | 1 | 1 | X | X | X | X | X | X | 0xXX | 6 bit BCD |
| | | | | | | | | | | data** |

Remarks:
*The Ack should be sent to acknowledge all of the commands sent by the device 205. If the commands sent by the device 205 expect other responses (e.g. Data), the Ack is not required.
**The data is encoded in Binary-Coded-Decimal. E.g., 0x10 = 0.10%, 0x19 = 0.19%. The maximum value is 0.19%.

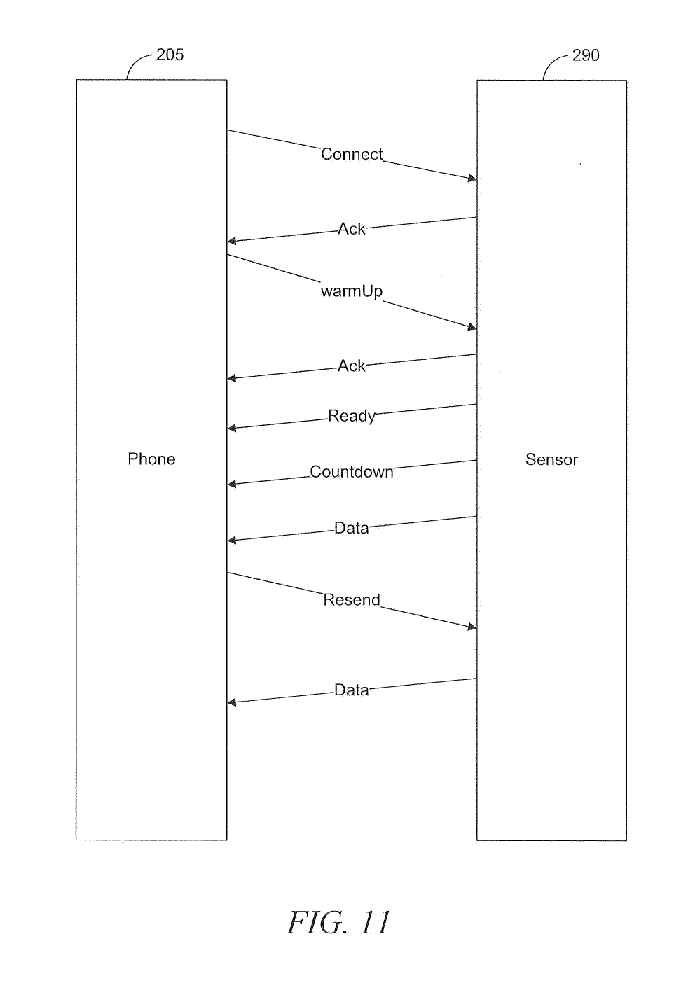

An example communication between the device 205 and the device 290 is shown in FIG. 11. In this example, the device 205 is labeled “Phone” and the device 290 is labeled “Sensor.”

In the sample communication sequence, the first Data block is missed. After time out, the App will send the Resend command and the device should reply with the last communication, which is Data.

The audio jack communication module 270 may communicate with audio jack communication module 260 via an audio jack. The audio jack communication module 270 may communicate with the sensor module 280. In some example embodiments, multiple sensor modules 280 are present. For example, a temperature sensor, a humidity sensor, and an ethanol sensor may be present. The audio jack communication module 270 may send data packets containing representations of sensor values generated by the sensor modules 280 to audio jack communication module 260. The audio jack communication, module 270 may receive commands from audio jack communication module 260. Based on commands received, the audio jack communication module 270 may control power provided to the sensor module 280, request a sensor reading from the sensor module 280, retransmit a packet to the audio jack communication module 260, or any suitable combination thereof.

The sensor module 280 may include a gas sensor. The gas sensor may detect a property (e.g., pressure, volume, temperature, mass, chemical composition, resistance, opacity, color, degree of contamination, or any suitable combination thereof) of a gas. The gas sensor may continuously monitor the gas, take readings only when requested, or both. The sensor module 280 may provide sensor readings to the audio jack communication module 270, receive commands from the audio jack communication module 270, or both. In some embodiments, the gas sensor may also provide error information to the audio jack communication module 270. For example, in an embodiment with a humidity sensor and a temperature sensor, if the humidity sensor only provides valid data within a range of temperatures, the sensor module 280 may provide an error value instead of a humidity value when the temperature is outside of the range of temperatures.

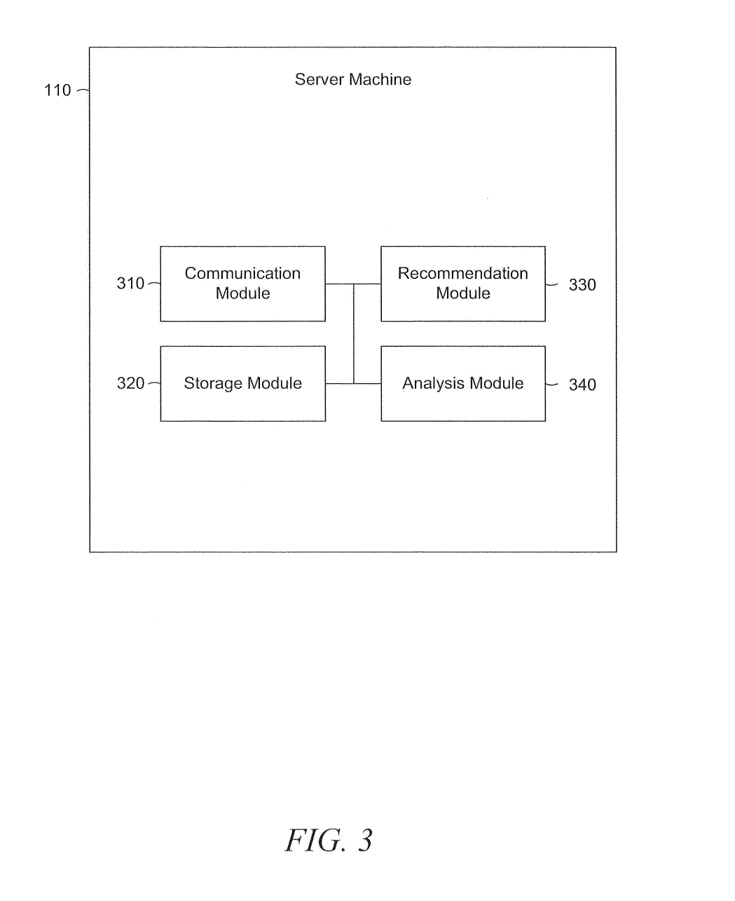

FIG. 3 is a block diagram illustrating components of the server machine 110, according to some example embodiments. The server machine 110 is shown as including a communication module 310, a storage module 320, a recommendation module 330, and an analysis module 340, all configured to communicate with each other (e,g., via a bus, shared memory, or a switch). Any one or more of the modules described herein may be implemented using hardware (e.g., a processor of a machine) or a combination of hardware and software. For example, any module described herein may configure a processor to perform the operations described herein for that module. Moreover, any two or more of these modules may be combined into a single module, and the functions described herein for a single module may be subdivided among multiple modules. Furthermore, according to various example embodiments, modules described herein as being implemented within a single machine, database, or device may he distributed across multiple machines, databases, or devices.

The communication module 310 may send data and commands from the server machine 110 to the user device 130. The communication module 310 may receive data and commands from the user device 130. In some example embodiments, the communication module 310 sends and receives data over the network 190. In some example embodiments, the communication module 310 sends data received from the user device 130 to the storage module 320. In some example embodiments, the communication module 310 sends data received from the user device 130 to the analysis module 340.

The storage module 320 may store and retrieve data from the database 115. The storage module 320 may process data before storing or retrieving it. For example, the storage module 320 may receive data in one format (e,g., binary-coded-decimal) and send it to the database 115 in another format (e.g., floating-point).

The recommendation module 330 may provide a recommendation based on data received from the communication module 310. For example, the communication module 310 may report data indicating that the user 132 has a BAC above the legal limit. In response, the recommendation module 330 may provide a recommendation that the user 132 call a cab instead of driving. In this example, recommendation module 330 provides the recommendation to the communication module 310, which transmits the recommendation to the user device 130. In some example embodiments, the recommendation module 330 may recommend that the user call a specific cab company. For example, the database 115 may store information regarding taxi companies in various areas. If the user device 130 reports that the user is in an area for which database 115 has information regarding taxi companies, recommendation module 330 may access the corresponding data and include the contact information of the taxi company with the recommendation. In some example embodiments, the choice of the taxi company to recommend is determined by an advertising fee paid by the taxi company. In other example embodiments, the choice of the taxi company to recommend is determined based on the shortest wait time for a cab.

In other example embodiments, the data received by the communications module 310 may relate to the carbon monoxide concentration at the location of the user device 130. In these example embodiments, the recommendation module 330 may provide a recommendation of increasing ventilation, evacuating the building, or both.

In other example embodiments, the data received by the communications module 310 may related to the concentration of pathogens at the location of the user device 130. In these example embodiments, the recommendation module 330 may provide a recommendation of contacting medical authorities, evacuating the area, quarantining the area, or any suitable combination thereof.

The analysis module 340 may perform analysis of data received by the communications module 310 or stored in the database 115. For example, after a number of users have reported their BACs, the analysis module 340 may analyze the aggregated data to determine patterns or trends of intoxication with respect to other collected data (e.g., location, population density, time of day, age of the individual, income of the individual, education level of the individual, or any suitable combination thereof).

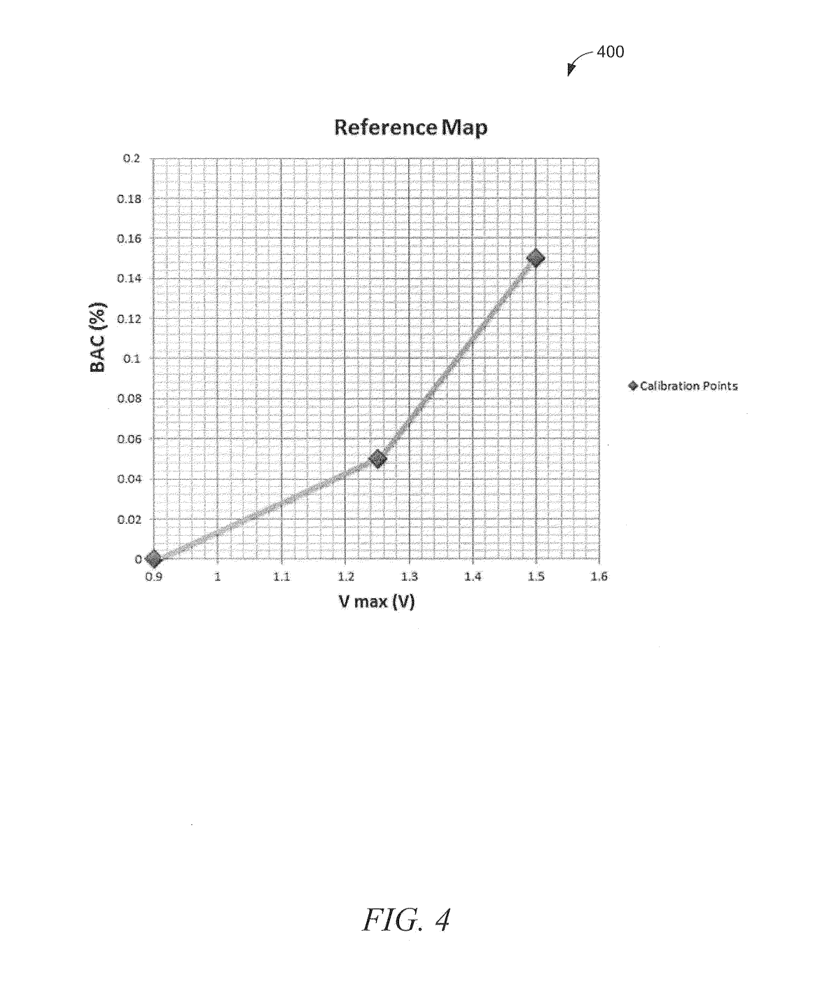

FIG. 4 is a graph illustrating a relationship between voltage and blood-alcohol content (“BAC”), according to some example embodiments. The x-axis of the graph 400 shows the maximum voltage output by an ethanol sensor. The y-axis of the graph 400 shows the BAC of an individual whose exhalations were tested by the sensor. The three calibration points, marked by diamonds, indicate known calibration values. In this case, the calibration values are 0% BAC at 0.9V, 0.05% BAC at 1.25V, and 0.15% BAC at 1.5V. This graph shows a linear interpolation between known values. In other example embodiments, higher-order interpolations (e.g., quadratic, cubic, etc.) may be used. In some example embodiments, more or fewer calibration points will be used, in some example embodiments, the graph will have multiple independent variables (e.g., two or more of a voltage, a temperature, a humidity).

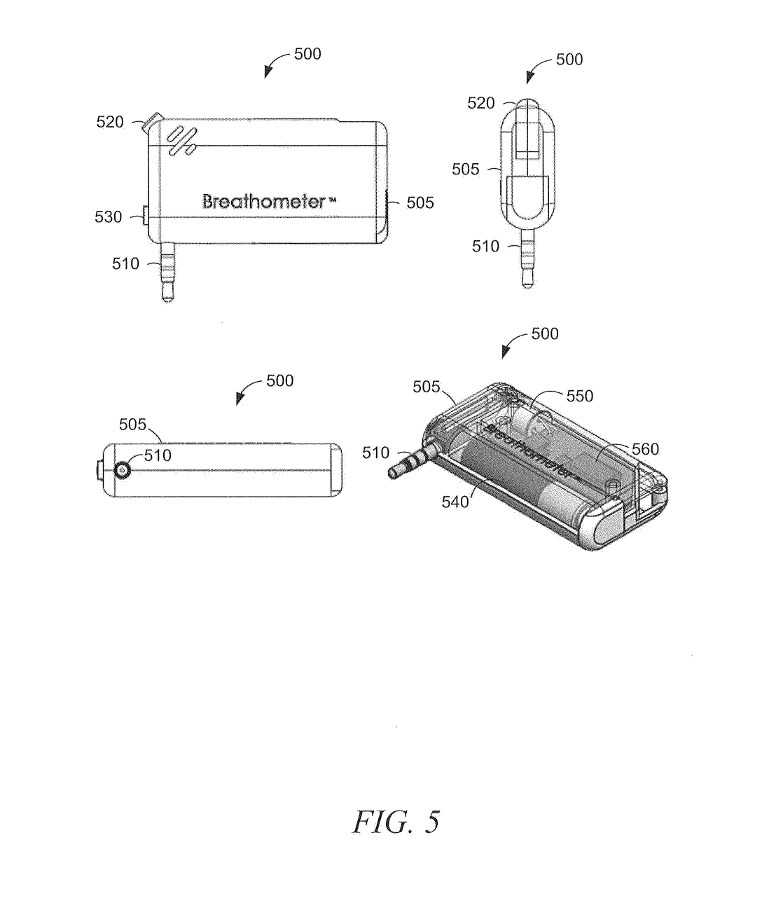

FIG. 5 is a diagram illustrating a device 500 capable of gathering gas data, according to some example embodiments. The device 500 may correspond to the device 290. Depicted are a housing 505, an audio jack 510, a gas intake port 520, a gas output port 530, a battery 540, a gas sensor 550, and an integrated circuit (IC) 560.

The housing 505 may be used to contain the battery 540, the gas sensor 550, the IC 560, and any other components of the device 500. In some example embodiments, the housing 505 protects its contents. In some example embodiments, the housing 505 is constructed from plastic, metal, or both. In some example embodiments, the housing is completely or partially transparent.

The audio jack 510 may he used to send data gathered by the gas sensor 550 to another device. In some example embodiments, the audio jack 510 is used to transmit power to the gas sensor 550, the IC 560, and any other components of the device 500. In such embodiments, the battery 540 may be absent or may be recharged by power received via the audio jack 510. For example, the audio jack 510 may be controlled by the audio jack communication module 270 and send data to be received by the audio, jack communication module 260. The audio jack 510 may protrude from the housing 505. In other example embodiments, the audio jack 510 may receive electronic signals sent by the audio jack communication module 260, and provide those signals to the audio jack communication module 270 for interpretation. In some example embodiments, the audio jack 510 has a tip-sleeve (“TS”) configuration, a tip-ring-sleeve (“TRS”) configuration, or a tip-ring-ring-sleeve (”TRRS”) configuration. For example, in a TRRS configuration, the sleeve may be grounded, the tip may provide power to the device 500, and each ring may provide a data channel,

The has intake port 520 may be used to receive the gas to be analyzed. For example, a user 132 may blow air directly into gas intake port 520. In another example, a user 132 may wave the device through the air to push air through the gas intake port 520. In other example embodiments, a tube, a fan, a vacuum device, or any suitable combination thereof may be used in conjunction with the gas intake port 520 to guide the gas to be tested to the gas sensor 550. In some example embodiments, a valve is used in conjunction with the gas intake port 520 to control the flow of gas. The gas intake port 520 may be an integral part of the housing 505 or a separate component. In some example embodiments, a tube is connected to the gas intake port 520. The gas may be blown through the tube into the gas intake port 520. Alternatively, the gas intake port 520 may be connected to a side of the tube and a pump used to siphon gas passing through the tube into the gas intake port 520. In some example embodiments, a flow sensor may be used to detect how much gas has passed through the tube, the gas intake port 520, or both. In some example embodiments, the gas sensor 550 does not begin sensing the property of the gas until a minimum amount of gas has been passed through the tube, the gas intake port 520, or both. The gas sensor 550 may stop sensing the property of the gas after a maximum amount of gas has been passed through the tube, the gas intake port 520, or both. In some example embodiments, the first portion of gas may have higher levels of undesired contamination and better results may be obtained by ignoring the first portion of the gas. For example, in a BAC tester, it may be desirable to ignore the first liter of gas exhaled. The amount of gas to be ignored may depend on a number of factors, such as the gender of the individual, the weight of the individual, the age of the individual, or any suitable combination thereof. For example, an average female has a lower total lung, capacity than an average male, and thus better results may be obtained by ignoring a greater volume of as from a male than from a female. As used herein, “active testing” refers to testing performed when the flow of gas from the location to be tested to the sensor is controlled. An example of active testing is when a user breathes into a tube that guides the exhaled air to the gas sensor 550. In contrast, “passive testing” refers to testing performed without such control of the flow of the gas. An example of passive testing is when a user exhales about one inch away from the as intake port 520. In this example, the user is forcing gas over the gas sensor 550, but the as to which the gas sensor 550 is exposed is a mixture of the exhaled air and other air from the environment.

The gas output port 530 may be used to exhaust the gas after the gas sensor 550 has been exposed to the gas. In some example embodiments, a valve is used in conjunction with the gas output port 530 to control the flow of gas. The gas output port 530 may be an integral part of the housing 505 or a separate component.

The battery 540 may be used to provide power to the gas sensor 550 and the IC 560. In some example embodiments, the battery 540 is easily replaceable by the user. In other example embodiments, the battery 540 is integrated into the device 500. In such example embodiments, after the battery 540 dies, the device 500 may be disposed of recycled, or both. In some example embodiments, the battery 540 is a standard AA or AAA battery. In some example embodiments, the battery 540 is a watch battery. In some example embodiments, the battery 540 is rechargeable. The battery 540 may be mounted directly to the housing 505, mounted to another component within the housing 505, or both.

The gas sensor 550 may determine a property of a was to which it is exposed. In some example embodiments, the gas sensor 550 provides an Output voltage indicating the property. For example, the gas sensor 550 may detect the electrical resistance of the gas, and output a higher voltage when a higher resistance is detected. In other example embodiments, the gas sensor 550 may detect a concentration of ethanol, carbon monoxide, pathogen, or other chemical or biological compound in the gas. In such example embodiments, the gas sensor 550 may output a voltage indicative of the concentration of the compound. The gas sensor 550 may be mounted directly to the housing 505, mounted to another component within the housing 505, or both. In some example embodiments, the gas Sensor 550 is a tin oxide (SnO₂) sensor, specific to ethanol sensing. In some example embodiments, the gas sensor 550 is optimized for a range near 0.05% BAC. In some, example embodiments, the tolerance of the gas sensor 550 is +/−0.02 at 0.1% BAC.

The IC 560 may control the gas sensor 550. The IC 560 may receive data from the gas sensor 550. In some example embodiments, the IC 560 and the gas sensor 550 are integrated into a single component. In some example embodiments, IC 560 is a dedicated hardware unit. In other example embodiments, IC 560 includes a general-purpose processor configured by software. The software may be stored in non-volatile memory within IC 560, on a separate IC, or both. The IC 560 may implement the sensor module 280, the audio jack communication module 270, or both. The IC 560 may implement these modules alone or in combination with the other elements of the device 500. The IC 560 may control output on the audio jack 510. The IC 560 may receive input from the audio jack 510. The IC 560 may be mounted directly to the housing 505, mounted to another component within the housing 505, or both. The IC 560 May receive a number of samples each second from the gas sensor 550. The IC 560 may s,ore, the highest sensor voltage received in a given period of time. The IC 560 may use an algorithm that compares the highest sensor voltage received to a data table stored in on board memory. The IC 560 may report a BAC based on the highest sensor voltage. The table mapping the highest sensor voltage to the BAC may be specific to individual gas sensor 550, and have been created via calibration. In some example embodiments, the calibration points are at 0.05% BAC and 0.1.5% BAC. The IC 560 may communicate with the gas sensor 550 via an application protocol interface (API).

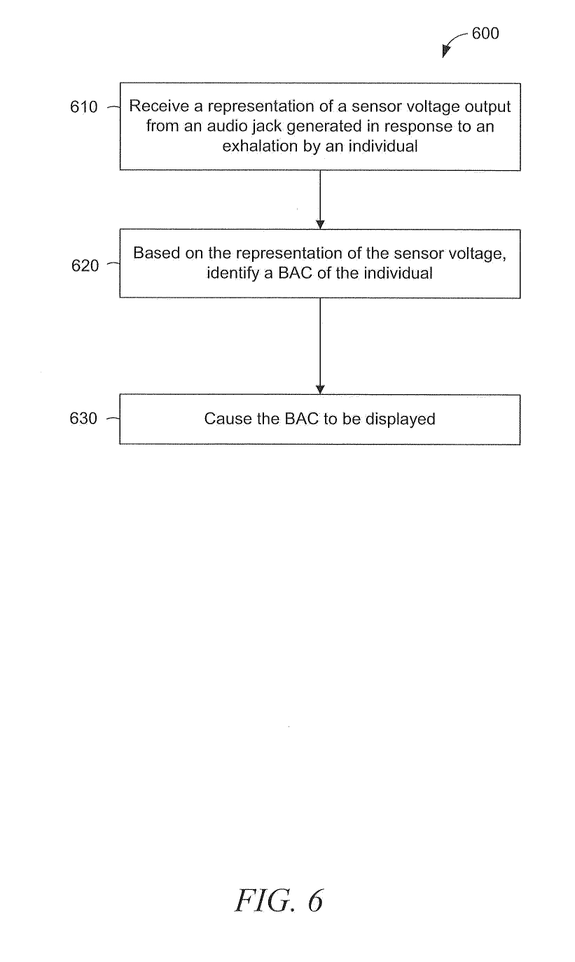

FIG. 6 is a flowchart illustrating operations of the device 205 in performing a method 600 of determining and presenting a user's BAC, according to some example embodiments. Operations in the method 600 may be performed by the device 205, using modules described above with respect to FIG. 2. As shown in FIG. 6, the method 600 includes operations 610, 620, and 630.

In operation 610, the audio jack communication module 260 receives a representation of a sensor voltage output from an audio jack generated in response to an exhalation by an individual. The individual may have exhaled into the gas input port 520. The sensor voltage may have been generated by the gas sensor 550. The sensor voltage may have been converted into a representation of the sensor voltage by the IC 560. The audio jack communication module 270 may have transmitted the representation of the sensor voltage via audio jack 510.

In operation 620, the analysis module 230 identifies a BAC of the individual based on the representation of the sensor voltage. In some example embodiments, the BAC of the individual is found by using a lookup table. For example, a table may be constructed in which each possible sensor voltage value provides a corresponding BAC. In some example embodiments, a multidimensional lookup table is used, with additional data used to determine a BAC for a particular sensor voltage value. For example, the BAC may vary based on the gender of the individual, the weight of the individual, the temperature of the gas, the humidity of the gas, or any suitable combination thereof. In some example embodiments, the BAC of the individual is found by using an equation. In such example embodiments, the BAC corresponding to the sensor voltage value may be calculated each time the sensor is read, rather than being stored in a lookup table. In some example embodiments, the BAC of the individual is determined on a different computing device, e.g., server machine 110. In such example embodiments, network communication module 310 may communicate the sensor voltage value to the server machine 110 and receive the BAC of the individual in response.

In operation 630, the display module 250 causes the BAC of the individual to be displayed. In sonic example embodiments, the BAC is displayed in numerical form. For example, a BAC of 0.08% may be represented by the text “0.08” on screen. In some example embodiments, the BAC is displayed as a point on a graph. In some example embodiments, the BAC is displayed as a boolean value resulting from the comparison of the BAC to a threshold value. For example, if the legal driving limit is 0.08, a portion of the screen may turn green if the BAC is below the limit and red if the BAC is above the limit. In another example embodiment, the information is displayed in text form. For example, if the BAC is below the limit, the string “Alcohol has been detected, but you are not over the limit” may be displayed, while if the BAC were above the limit, the string “Get a friend to drive” may be displayed. In some example embodiments, the device 205 is a smart phone and the display module 250 causes the BAC to be displayed on the screen of the smart phone.

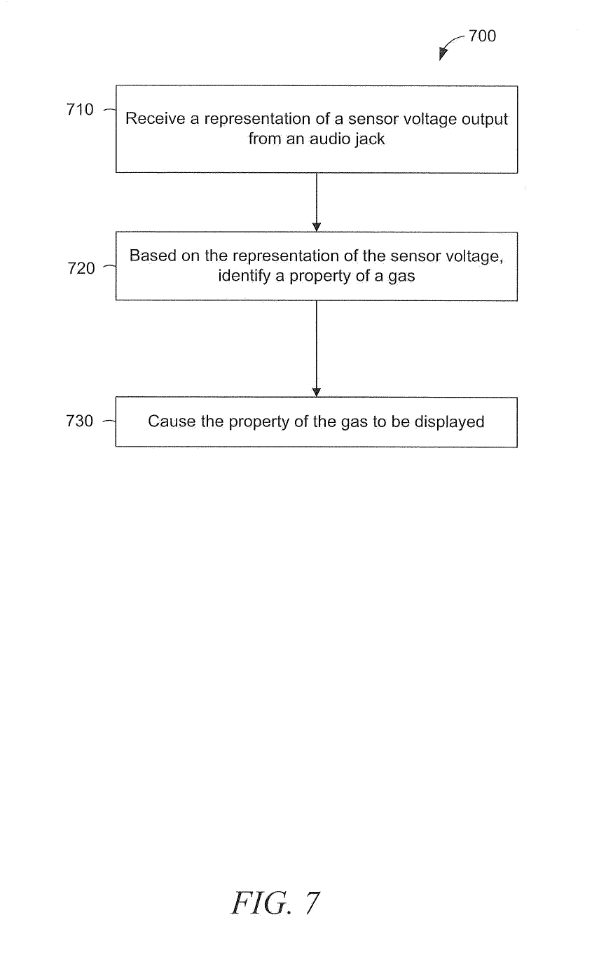

FIG. 7 is a flowchart illustrating operations of the device 205 in performing a method 700 of determining and presenting a property of a gas, according to some example embodiments. Operations in the method 700 may be performed by the device 205, using modules described above with respect to FIG. 2. As shown in FIG. 7, the method 700 includes operations 710, 720, and 730.

In operation 710, the audio jack communication module 260 receives a representation of a sensor voltage output from an audio jack generated in response to exposure to a gas. The gas may have entered the device 500 via the gas input port 520. The sensor voltage may have been generated by the gas sensor 550. The sensor voltage 550 may have been converted into a representation of the sensor voltage by the IC 560. The audio jack communication module 270 may have transmitted the representation of the sensor voltage via audio jack 510.

In operation 720, the analysis module 230 identifies a property of the gas based on the representation of the sensor voltage. In some example embodiments, the property of the gas is found by using a lookup table. For example, a table may be constructed in which each possible sensor voltage value provides a corresponding property of the gas. In some example embodiments, a multidimensional lookup table is used, with additional data used to determine a property of the gas for a particular sensor voltage value. The property of the gas may vary based on the temperature of the gas, the humidity of the gas, the GPS location at which the sensor was exposed to the gas, or any suitable combination thereof. In some example embodiments, the property of the gas is found by using an equation. In such example embodiments, the property of the gas corresponding to the sensor voltage value may be calculated each time the sensor is read, rather than being stored in a lookup table, in some example embodiments, the property of the gas is determined on a different computing device, e.g., server machine 110. In such example embodiments, network communication module 310 may communicate the sensor voltage value to the server machine 110 and receive the property of the gas in response.

In operation 730, the display module 250 causes the property of the gas to be displayed. In some example embodiments, the property of the gas is displayed in numerical form. For example, a carbon monoxide concentration of 5 parts per million (ppm) may be represented by the text “5” on screen. In some example embodiments, the property of the gas is displayed as a point on a graph. In some example embodiments, the property of the gas is displayed as a boolean value resulting from the comparison of the property to a threshold value. For example, if a toxicity level of carbon monoxide is 100 ppm, a portion of the screen may turn green if the carbon monoxide concentration is below the limit and red if the carbon monoxide concentration is above the limit. In another example embodiment, the information is displayed in text form. For example, if the property is below the limit, the string “Air OK” may be displayed, while if the property were above the limit, the string “Air Toxic” may be displayed. In some example embodiments, the device 205 is a smart phone and the display module 250 causes the property to be displayed on the screen of the smart phone.

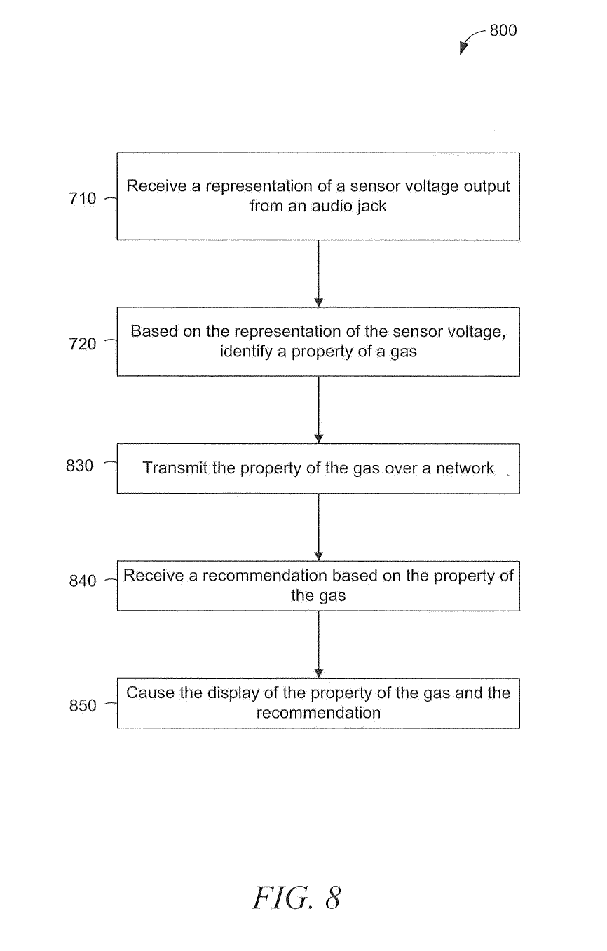

FIG. 8 is a flowchart illustrating operations of the device 205 in performing a method 800 of determining and presenting a property of a gas, according to some example embodiments. Operations in the method 800 may be performed by the device 205, using modules described above with respect to FIG. 2. As shown in FIG. 8, the method 800 includes operations 710, 720, 830, 840, and 850. Operations 710 and 720 have been described above with respect to FIG. 7.

In operation 830, the device 205, via network communications module 210, may send the property of the gas determined in operation 720 to the server machine 110In alternative embodiments, data derived from the property of the gas may be sent to the server machine 110 in addition to or instead of the property of the gas itself.

In operation 840, the device 205, via the network communications module 210, may receive a recommendation based on the property of the gas from the server machine 110. For example, if the property of the gas is the concentration of carbon monoxide in the gas, the recommendation may recommend evacuating the area if the carbon monoxide concentration is high.

In operation 850, the device 205, via the display module 250, may cause the display the property of the as and the recommendation received front the server machine 110. For example, if the property of the gas is a concentration of 110 ppm carbon monoxide, the recommendation may be to evacuate the building, and both “110 ppm” and “Evacuate” may be displayed. As described above with respect to operation 730, the property and the recommendation may be displayed in various ways such as numerical, text, color, or any suitable combination thereof.

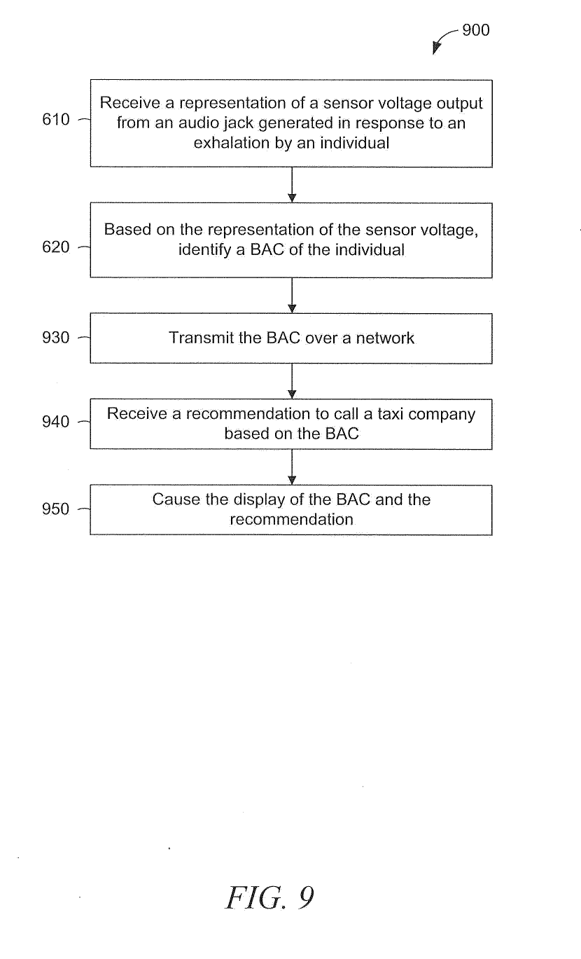

FIG. 9 is a flowchart illustrating operations of the device 205 in performing a method 900 of determining and presenting a property of a gas, according to some example embodiments. Operations in the method 900 may be performed by the device 205, using modules described above with respect to FIG. 2. As shown in FIG. 9, the method 900 includes operations 610, 620, 930, 940, and 950. Operations 610 and 620 have been described above with respect to FIG. 6.

In operation 930, the device 205, via network communications module 210, may send the BAC determined in operation 620 to the server machine 110.

In operation 940, the device 205, via the network communications module 210, may receive a recommendation based on the BAC from the server machine 110. The recommendation may be a recommendation to call a taxi company. For example, if the BAC of the individual exceeds the legal limit, the device 205 may receive a recommendation to call a nearby taxi company. The taxi company may be recommended based on proximity to the individual, availability of cabs, a fee paid by the taxi company, or any suitable combination thereof.

In operation 950, the device 205, via the display module 250, may cause the display of the BAC of the individual and the recommendation received from the server machine 110. The BAC may be displayed in a variety of forms, including numerical, text, and graphical, as described above with respect to operation 630. The recommendation to call a taxi company may also be displayed in a variety of forms. For example, the recommendation may appear in text “Please call Taxi Company X at (555) 555-1234.” Or, if the device 205 is capable of placing telephone calls, a button or other user interface element may be displayed that, when activated, calls the taxi company.

According to various example embodiments, one or more of the methodologies described herein may facilitate identification of a property of a gas. In some example embodiments, the use of a portable device 290 in combination with a processing device 205 is more convenient and less expensive than the use of a monolithic device capable of identifying a property of a gas. In some example embodiments, the user device 130 in combination with the server device 110 is more convenient and less expensive than the use of a monolithic device capable of identifying a property of a gas. In some example embodiments, the use of an audio jack to communicate data between the device 205 and the device 290 is more convenient and more efficient than using a traditional data connection to connect the devices.

When these effects are considered in aggregate, one or more of the methodologies described herein may obviate a need for certain efforts or resources that otherwise would be involved in identifying properties of gases. Efforts expended by a user in identifying a property of a gas may be reduced by one or more of the methodologies described herein. Computing resources used by one or more machines, databases, or devices (e.g., within the network environment 100) may similarly be reduced. Examples of such computing resources include processor cycles, network traffic, memory usage, data storage capacity, power consumption, and cooling capacity.

FIG. 10 is a block diagram illustrating components of a machine 1000, according to some example embodiments, able to read instructions from a machine-readable medium (e.g., a machine-readable storage medium, a computer-readable storage medium, or any suitable combination thereof) and perform any one or more of the methodologies discussed herein, in whole or in part. Specifically, FIG. 10 shows a diagrammatic representation of the machine 1000 in the example form of a computer system and within which instructions 1024 (e.g., software, a program, an application, an applet, an app, or other executable code) for causing the machine 1000 to perform any one or more of the methodologies discussed herein may be executed, in whole or in part. In alternative embodiments, the machine 1000 operates as a standalone device or may be connected (e,g., networked) to other machines. In a networked deployment, the machine 1000 may operate in the capacity of a server machine or a client machine in a server-client network environment, or as a peer machine in a distributed (e.g., peer-to-peer) network environment. The machine 1000 may be a server computer, a client computer, a personal computer (PC), a tablet computer, a laptop computer, a netbook, a set-top box (STB), a personal digital assistant (PDA), a cellular telephone, a smartphone, web appliance, a network router, a network switch, a network bridge, or any machine capable of executing the instructions 1024, sequentially or otherwise, that specify actions to be taken by that machine. Further, while only a single machine is illustrated, the term “machine” shall also be taken to include a collection of machines that individually or jointly execute the instructions 1024 to perform all or part of any one or more of the methodologies discussed herein.

The machine 1000 includes a processor 1002 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 1004, and a static memory 1006, which are configured to communicate with each other via a bus 1008. The machine 1000 may further include a graphics display 1010 (e.g., a plasma display panel (PDP), a light emitting diode (LED) display, a liquid crystal display (LCD), a projector, or a cathode ray tube (CRT)). The machine 1000 may also include an alphanumeric input device 1012 (e.g., a keyboard), a cursor control device 1014 (e,g., a mouse, a touchpad, a trackball, a joystick, a motion sensor, or other pointing instrument), a storage unit 1016, a signal generation device 1018 (e.g., a speaker), and a network interface device 1020.

The storage unit 1016 includes a machine-readable medium 1022 on which is stored the instructions 1024 embodying any one or more of the methodologies or functions described herein. The instructions 1024 may also reside, completely or at least partially, within the main memory 1004, within the processor 1002 (e.g., within the processor's cache memory), or both, during execution thereof by the machine 1000. Accordingly, the main memory 1004 and the processor 1002 may be considered as machine-readable media. The instructions 1024 may be transmitted or received over a network 1026 (e.g., network 190) via the network interface device 1020.

As used herein, the term “memory” refers to a machine-readable medium able to store data temporarily or permanently and may he taken to include, but not be limited to, random-access memory (RAM), read-only memory (ROM), buffer memory, flash memory, and cache memory. While the machine-readable medium 1022 is shown in an example embodiment to be a single medium, the term “machine-readable medium” should be taken to include a single medium or multiple media (e.g., a centralized or distributed database, or associated caches and servers) able to store instructions. The term “machine-readable medium” shall also be taken to include any medium, or combination of multiple media, that is capable of storing instructions for execution by a machine (e,g., machine 1000), such that the instructions, when executed by one or more processors of the machine (e.g., processor 1002), cause the machine to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” shall accordingly be taken to include, but not be limited to, one or more data repositories in the form of a solid-state memory, an optical medium, a magnetic medium, or any suitable combination thereof.

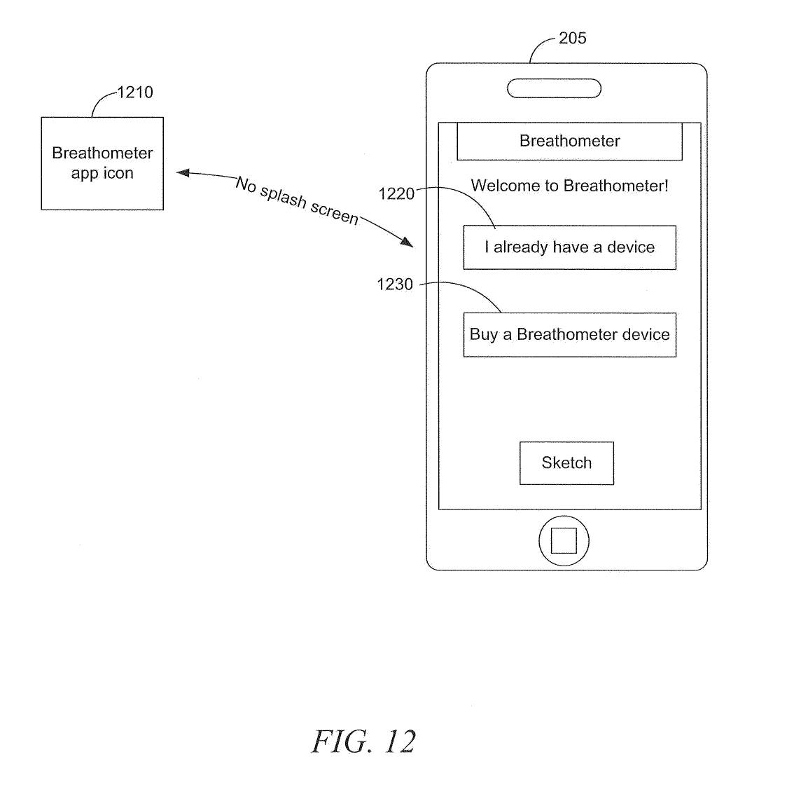

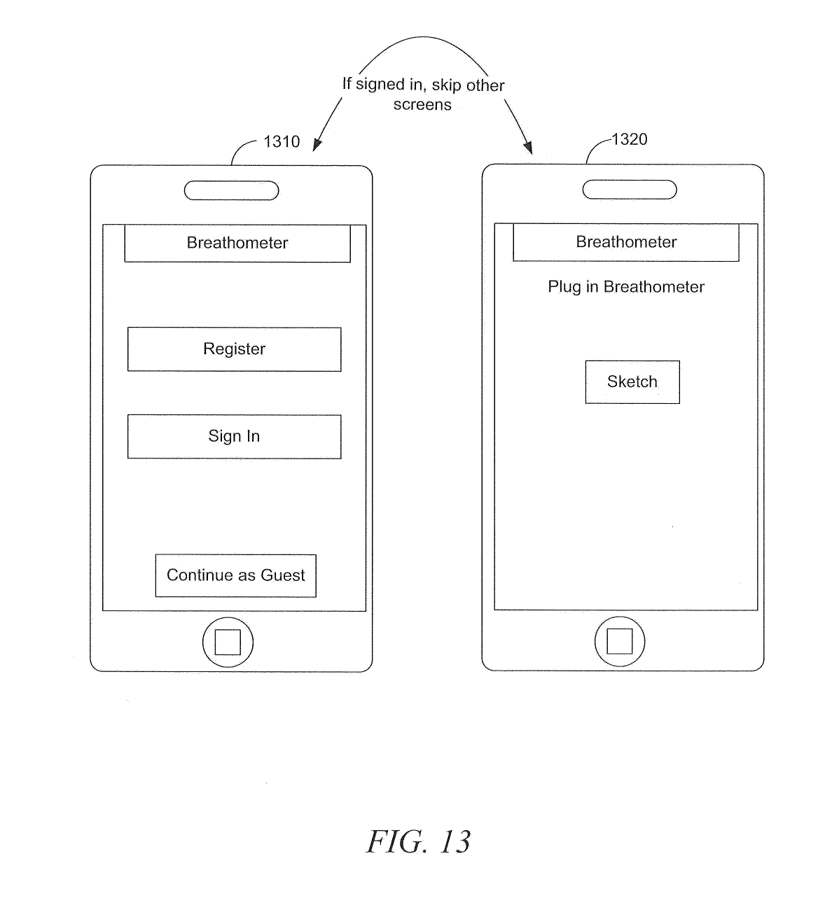

In FIG. 12 a mobile computing device 205, such as a smart phone, is shown. ‘ibis device may correspond to the device 205. When the user activates the Breathometer App Icon 1210, the Breathometer application [may he launched. On this screen, the user is presented with two buttons 1220 and 1230. The button 1220, labeled, “I already have a device,” may be operable to inform the application that a user device 290 is ready for use with the mobile computing device. The button 1230, labeled, “Buy a Breathometer device,” may be operable to direct the user to an electronic commerce site from which a gas sensor device 500 may be purchased. In FIG. 13, two more screens 1310 and 1320 of a UI are shown. In the screen 1310, the first button, labeled “Register,” may be operable to register the user device 130 with the server machine 110. The second button, labeled “Sign In,” may be operable by an already-registered user to connect with the server machine 110. In some example embodiments, once a user is signed in, the device will sign in automatically when the application is launched. In the screen 1320, the application has detected that no device 500 is connected to the smart phone. Accordingly, a “Plug in Breathometer” message is displayed, prompting the user to connect the device 500.

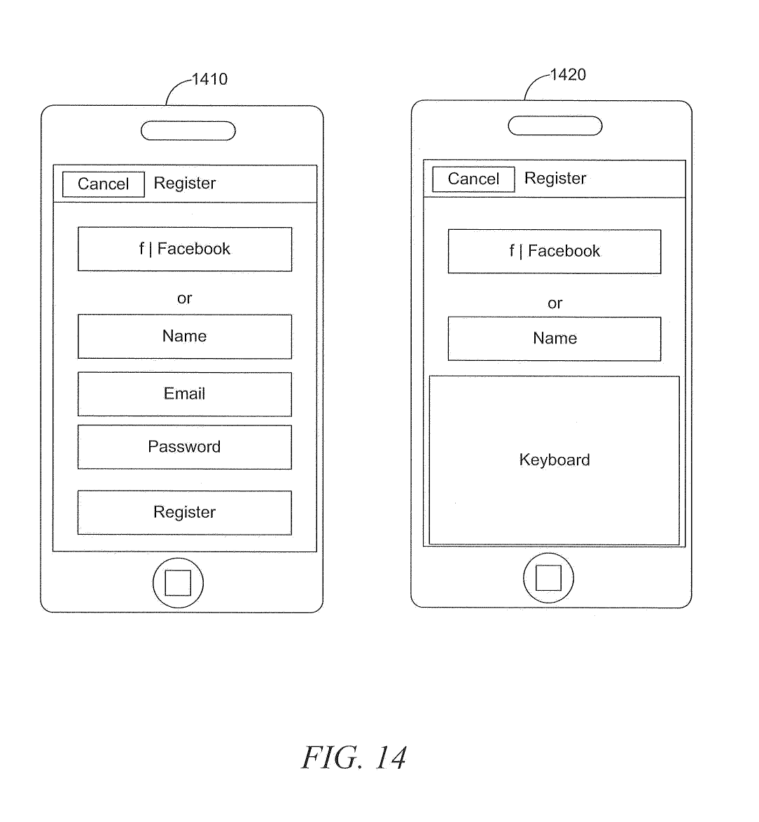

In FIG. 14, two more screens 1410 and 1420 of a UI are shown. These screens may be displayed in response to the operation of the “Register” button described above. In some example embodiments, a user may register the application and device via an existing social media account (e.g., Facebook, Twitter, MySpace). In some example embodiments, the application supports registration via an independent account. In the screen on the left, the user is presented with a “Cancel” button, a “Facebook” button, “Name,” “Email,” and “Password” text fields, and a “Register” button. The “Cancel” button may be operable to cancel the registration process. The “Facebook” button may be operable to register using information from an existing social media account. The text fields may be operable to accept information identifying the user. The “Register” button may be operable to register the user using information entered into the text fields. The screen 1420 shows a virtual keyboard of a smart phone device. The virtual keyboard may be used to enter text into the text fields.

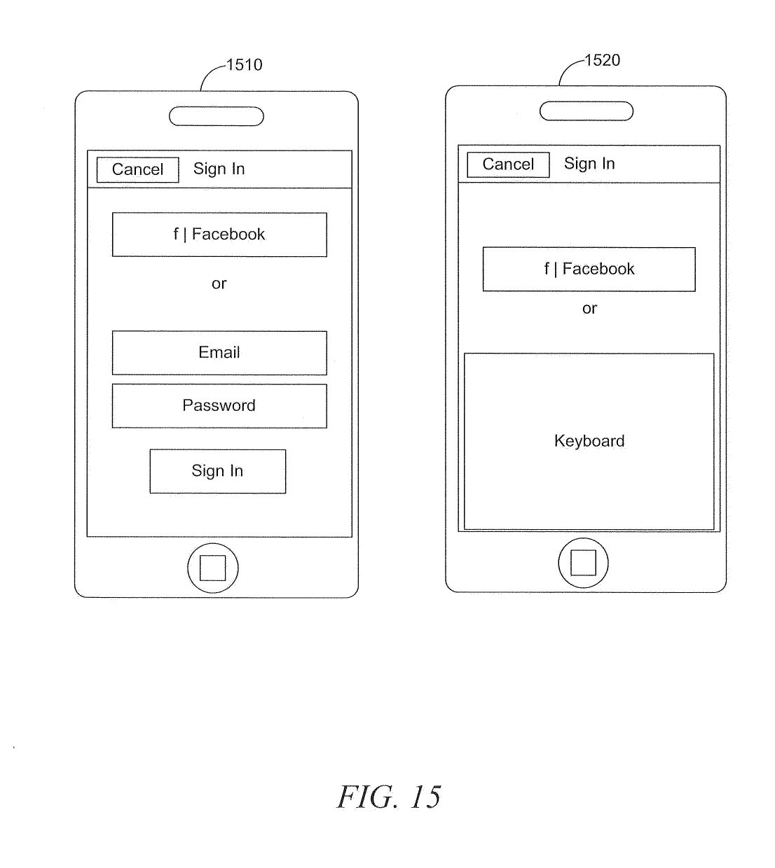

In FIG. 15, two more screens 1510 and 1520 of a UI are shown. These screens may be displayed in response to the operation of the “Sign In” button described above. In some example embodiments, the application supports registration via an independent account. In the screen 1410, the user is presented with a “Cancel” button, a “Facebook” button, “Email,” and “Password” text fields, and a “Sign In” button. The “Cancel” button may be operable to cancel the sign in process. The “Facebook” button may be operable to sign in using information from an existing social media account. The text fields may be operable to accept independent account information for the user. The “Sign In” button may be operable to sign in the user using information entered into the text fields. The screen 1520 shows a virtual keyboard of a smart phone device. The virtual keyboard may he used to enter text into the text fields.

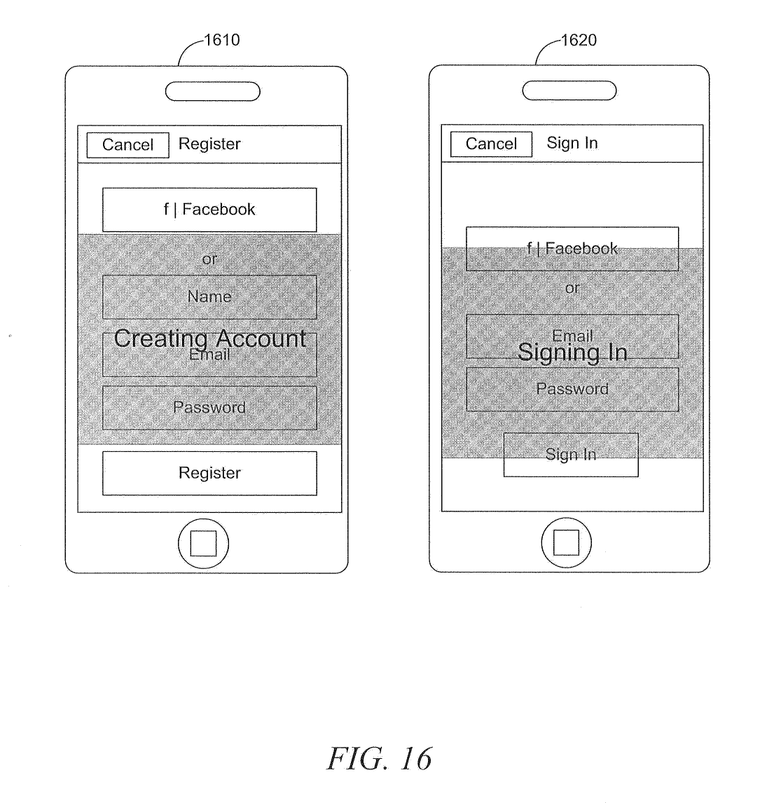

In FIG. 16, two more screens 1610 and 1620 of a UI are shown. The screen 1610 may be displayed in response to the operation of the “Facebook” or “Register” buttons discussed above. The screen 1620 may be displayed in response to the operation of the “Facebook” or “Sign In” buttons discussed above. The screen 1610 may inform the user that account creation is taking place, for example by displaying the text “Creating Account” The screen 1620 may inform the user that account validation is taking place, for example by displaying the text “Signing In.” In some example embodiments, one of the screens 1610 or 1620 is displayed while the user device 130 is communicating with the server machine 110. Displayed on each of the two screens is a “Cancel” button. In some example embodiments, the “Cancel” button is operable to cancel the network communication. In other example embodiments, the “Cancel” button is inoperable until the network communication is complete or is aborted by the application. In some example embodiments, the server machine 110 compares the user credentials submitted with user credentials stored in the database 115. In some example embodiments, when a match is found, the user is logged in. In some example embodiments, when a match is not found, an error message is given to user. In some example embodiments, when a match is not found, the user prompted to sign in again. In other example embodiments, when a match is not found, the application allows the user to proceed with testing.

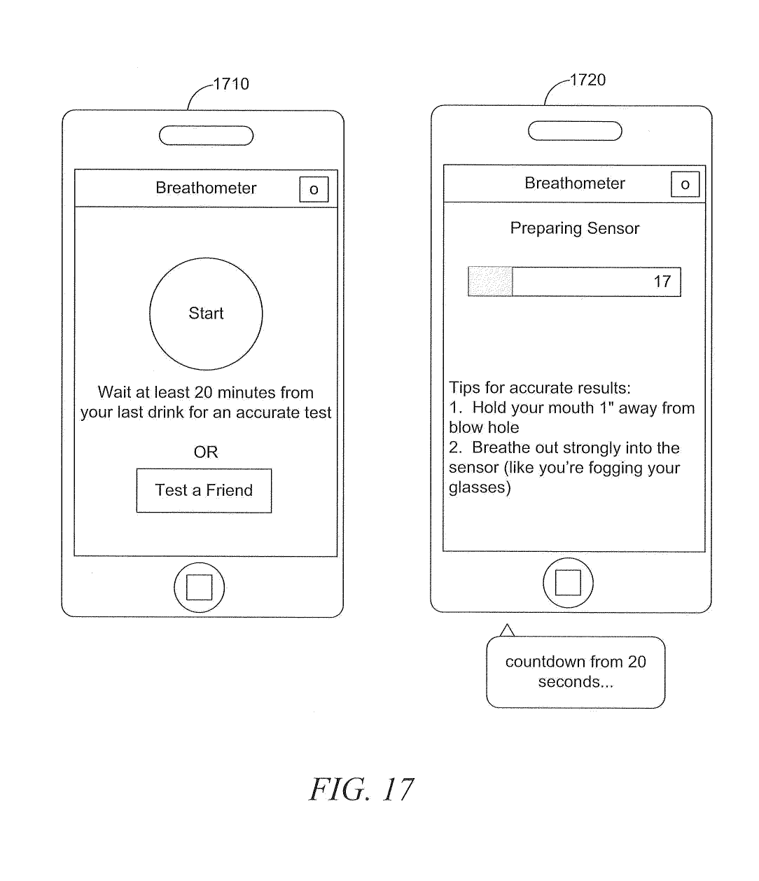

In FIG. 17, two more screens 1710 and 1720 of a UI are shown. In the screen 1710, a button labeled “Start” is presented. This button may be operable to begin a test. In some example embodiments, the results of tests started via the “Start” button are associated with the user who registered the device. A second button labeled “Test a Friend” is also presented. This button may also be operable to begin a test. In some example embodiments, the results of tests started via the “Test a Friend” button are not associated with any user. In other example embodiments, user data is gathered for the individual being tested.

In the screen 1720, a timer is shown as counting down from 20 seconds. In some example embodiments, a delay is required to prepare the gas sensor module for testing. In some example embodiments, the length of the required delay is 20 seconds. Also shown on the screen 1620 are recommendations for the proper use of the device.

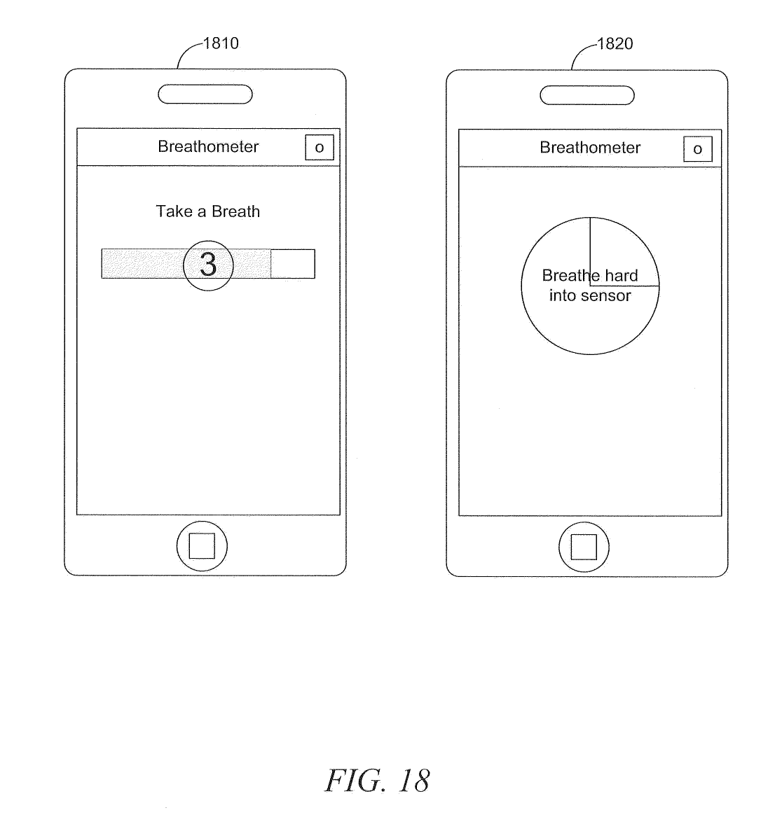

In FIG. 18, two more screens 1810 and 1820 of a UI are presented. In the screen 1810, instructions are presented to the user to prepare to exhale gas into the sensor. Also shown is a count-down timer. The count-down timer may count down to 0, to allow the user to predict when to start exhaling. In some example embodiments, a graphical representation of the count-down is used in addition to or instead of the numeric count down.

In the screen 1820, a prompt is shown that instructs the user to “Breathe hard into sensor.” Also shown is a circle. The circle may fill with color as the flow of air is detected, in some example embodiments, the circle completely fills with color when sufficient air for a valid test is detected. In some example embodiments, the circle completely fills with color after a set amount of time (e.g., 5 seconds) has passed.

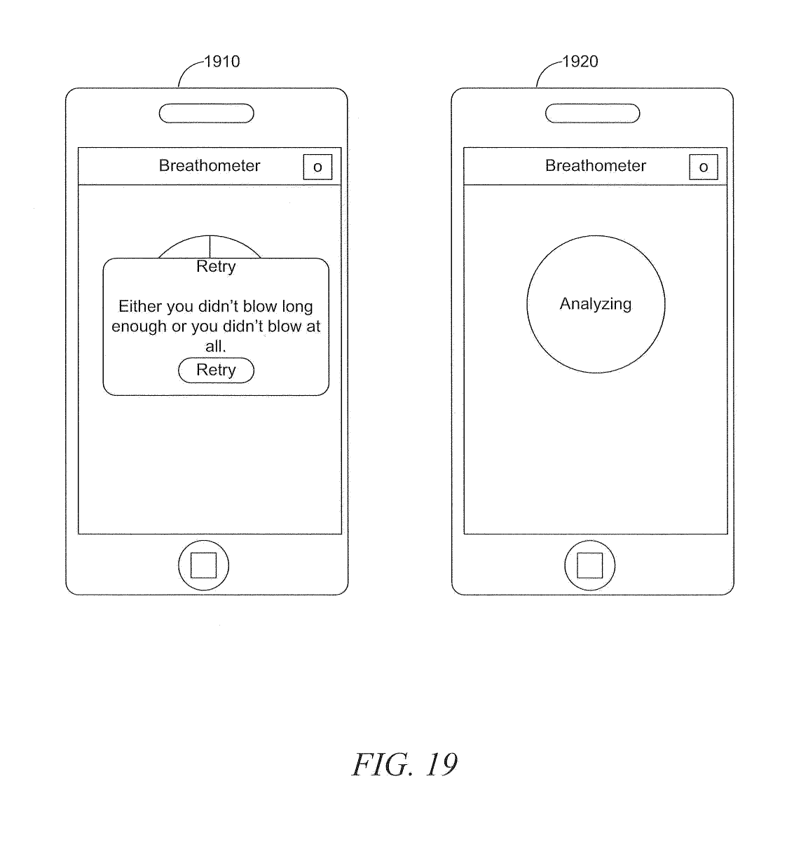

The circle may be shown filling with color as the animation ring completes its progress over 5 seconds (e.g., like a clock or pie chart). FIG. 19, two more screens 1910 and 1920 of a UI are shown. The screen 1910 may be displayed when insufficient air flow is detected. The button labeled “Retry” may be operable to restart the test. The screen 1920 may be displayed when sufficient air flow is detected. The “Analyzing” message indicates to the user that data from the gas sensor is being processed. In some example embodiments, an area around the Analyzing message is animated (e.g., pulsating, rotating, changing colors, or any suitable combination thereof) to keep the user's attention while the data is processed. The screen 1920 may be displayed for a fixed period of time, such as 1 second.

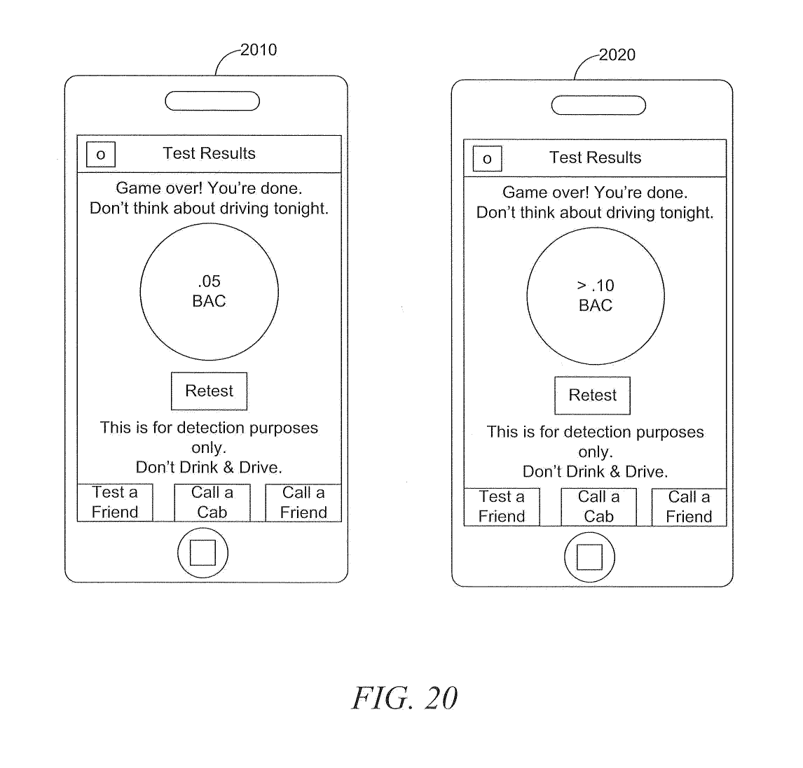

In FIG. 20, two more screens 2010 and 2020 of a are shown. In the screen 2010, a result of a test is shown as 0.05% BAC. Also shown are buttons labeled “Retest,” “Test a friend,” “Call a Cab,” and “Call a Friend.” The screen 2020 is similar, except the result of the test is shown as 0.10% BAC. In some example embodiments, the test results may be color-coded. For example, a 0% BAC may appear green while a 0.05% BAC appears yellow and a 0.10% BAC appears red. In some example embodiments, a BAC above the legal limit appears red, a BAC above 0 and below the legal limit appears yellow, and a BAC of 0 appears green. In some example embodiments, the “Retest” button is operable to start another test of the same individual. In some example embodiments, the “Test a Friend” button is operable to start a test of a different individual. In some example embodiments, the “Call a Cab” button is operable to call a taxi. In some example embodiments, the “Call a Friend” button is operable to open a phone application, start a phone call to a previously identified individual, or both. In some example embodiments, previous results are shown as well as current results. The set of results may be limited to tests taken within a specific time period (e.g., the last four hours).

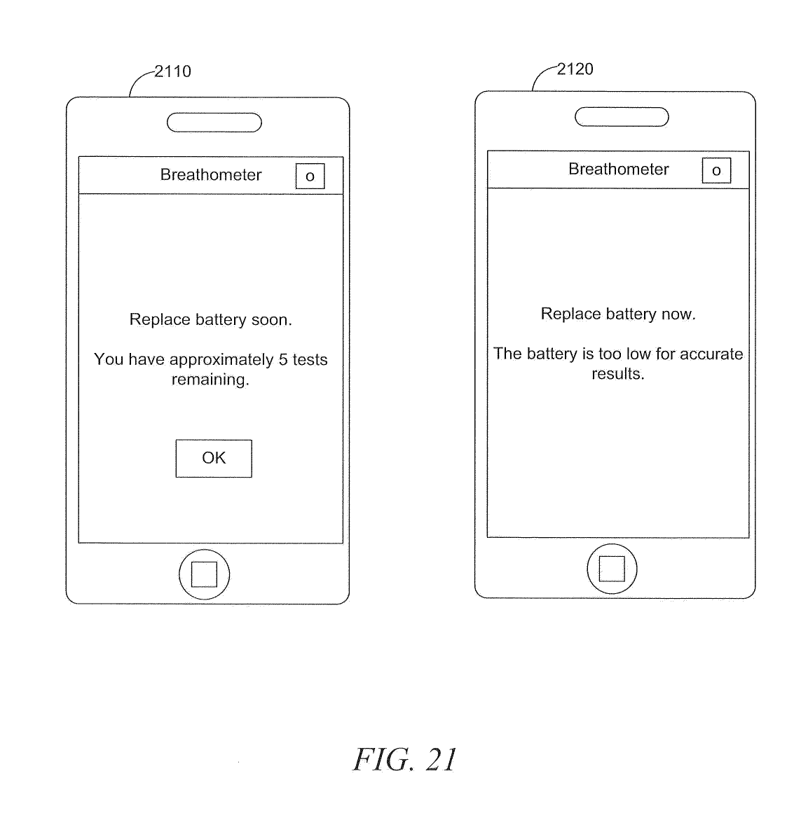

In FIG. 21, two more screens 2110 and 2120 of a UI are shown. In some example embodiments, the application detects when the battery 540 is low. The screen 2110 depicts a warning message indicating that the battery is low and that approximately 5 tests are remaining. A button labeled “OK” is also displayed. The “OK” button may be operable to dismiss the warning and proceed with a test. The screen 2020 depicts an error message indicating that the battery is too low for testing some example embodiments, a button operable to dismiss the error message may be presented. In other example embodiments, other UI elements (e.g., a hardware or software menu button) may be used to close the error screen, exit the application, or both. In some example embodiments, the application will detect when the battery is replaced and close the error screen in response. The user may not be able to proceed to test unless the battery is replaced.

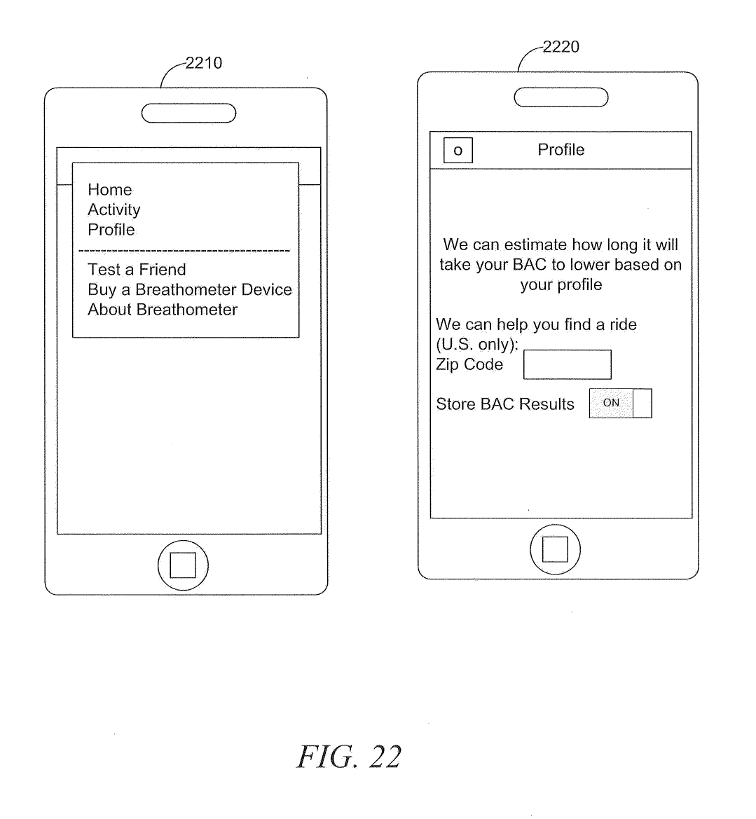

FIG. 22 shows two more screens 2210 and 2220 of a UI. In some example embodiments, the application provides a menu. In the screen 2210, the menu includes “Home,” “Activity,” “Profile,” “Tell a Friend,” “Buy a Breathometer Device,” and “About Breathometer” options. The “Home” option may be operable to return the user to a home screen of the application. For example, a screen including a button operable to start a test. The “Activity” option may be operable to provide a screen to the user displaying previous test results. In some example embodiments, the previous test result screen includes test results, user data, times and dates at which the tests took place, locations of the tests, or any suitable combination thereof. The “Profile” option may be operable to provide a screen to the user that allows the user to see and edit the user data associated with the user or the device. The “Tell a Friend” option may be operable to provide a screen that allows the user to send a message to friend recommending the gas sensor device or the application. For example, the message may be sent by phone, email, text message, social media, or any suitable combination thereof. The message may be composed by the user, generated by the application, or both. The “Buy a Breathometer Device” option may be operable to direct the user to an online merchant from whom the gas sensor device may be purchased. The “About Breathometer” option may be operable to provide a screen describing the gas sensor device, the company producing the device, the application, the company producing the application, or any suitable combination thereof.

The screen 2220 depicts a profile-editing screen, according to some example embodiments. The screen 2220 depicts a text field for receiving a zip code of a user. The zip code entered into the text field may be used to determine a taxi company to call when BAC levels exceed legal limits. The screen 2220 depicts a slider for allowing a user to choose whether to save BAC results or not. In some example embodiments, BAC results are stored only when the user's profile enables the storage. In other example embodiments, BAC results are stored without personally identifying information when the user has disabled storage. In some example embodiments, the user profile includes personal data such as height, weight, age, gender, medical history, occupation, or any suitable combination thereof.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein,

Certain embodiments are described herein as including logic or a number of components, modules, or mechanisms. Modules may constitute either software modules (e.g., code embodied on a machine-readable medium or in a transmission Signal) or hardware modules. A “hardware module” is a tangible unit capable of performing certain operations and may be configured or arranged in a certain physical manner. In various example embodiments, one or more computer systems (e.g., a standalone computer system, a client computer system, or a server computer system) or one or More hardware modules of a computer system (e.g., a processor or a group of processors) may be configured by software (e.g., an application or application portion) as a hardware module that operates to perform certain operations as described herein.

In some embodiments, a hardware module may be implemented mechanically, electronically, or any suitable combination thereof. For example, a hardware module may include dedicated circuitry or logic that is permanently configured to perform certain operations. For example, a hardware module may be a special-purpose processor, such as a field programmable gate array (FPGA) or an ASIC. A hardware module may also include programmable logic or circuitry that is temporarily configured by software to perform certain operations. For example, a hardware module may include software encompassed within a general-purpose processor or other programmable processor. It will be appreciated that the decision to implement a hardware module mechanically, in dedicated and permanently configured circuitry, or in temporarily configured circuitry (e.g., configured by software) may be driven by cost and time considerations.

Accordingly, the phrase “hardware module” should be understood to encompass a tangible entity, be that an entity that is physically constructed, permanently configured (e.g., hardwired), or temporarily configured (e.g., programmed) to operate in a certain manner or to perform certain operations described herein. As used herein, “hardware-implemented module” refers to a hardware module. Considering embodiments in which hardware modules are temporarily configured (e.g., programmed), each of the hardware modules need not be configured or instantiated at any one instance in time. For example, where a hardware module comprises a general-purpose processor configured by software to become a special-purpose processor, the general-purpose processor may be configured as respectively different special-purpose processors (e.g., comprising different hardware modules) at different times. Software may accordingly configure a processor, for example, to constitute a particular hardware module at One instance of time and to constitute a different hardware module at a different instance of time.

Hardware modules can provide information to, and receive information from, other hardware modules. Accordingly, the described hardware modules may be regarded as being communicatively coupled. Where multiple hardware modules exist contemporaneously, communications may he achieved through signal transmission (e.g., over appropriate circuits and buses) between or among two or more of the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware, modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions described herein. As used herein, “processor -implemented module” refers to a hardware module implemented using one or more processors.

Similarly, the methods described herein may be at least partially processor-implemented, a processor being an example of hardware. For example, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an application program interface (API)).

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of the subject matter discussed herein may be, presented in terms of algorithms or symbolic representations of operations on data stored as bits or binary digital signals within a machine memory (e.g., a computer memory). Such algorithms or symbolic representations are examples of techniques used by those of ordinary skill in the data processing arts to convey the substance of their work to others skilled in the art. As used herein, an “algorithm” is a self consistent sequence of operations or similar processing leading to a desired result. In this context, algorithms and operations involve physical, manipulation of physical quantities. Typically, but not necessarily, such quantities may take the form of electrical, magnetic, or optical signals capable of being stored, accessed, transferred, combined, compared, or otherwise manipulated by a machine, it is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., computer) that manipulates or transforms data represented as physical (e.g., electronic, magnetic, or optical) quantities within one or more memories (e.g., volatile memory, non-volatile memory, or any suitable combination thereof), registers, or other machine components that receive, store, transmit, or display information. Furthermore, unless specifically stated otherwise, the terms “a” or “an” are herein used, as is common in patent documents, to include one or more than one instance. Finally, as used herein, the conjunction “or” refers to a non exclusive “or,” unless specifically stated otherwise.

The following enumerated descriptions define various example embodiments of methods, machine-readable media, and systems (e.g., apparatus) discussed herein:

1. A device comprising:
a housing;
an intake port through which a gas can pass into the housing;
an electronic gas sensor disposed within the housing, the electronic gas sensor configured to generate a sensor voltage based on an amount of a substance detected in the gas;
a processor configured to convert the sensor voltage to a result value; and an audio jack protruding from the housing, the audio, jack communicatively coupled to the electronic gas sensor and configured to output the result value via a communications protocol.

2. The device of description 1, wherein
the result value is a representation of the sensor voltage,

3. The device of description 1, wherein
the result value represents a property of the gas.

4. The device of description 1, further comprising:
a processor configured to:
   receive the representation of the sensor voltage outputted from the audio jack; and
   determine a property of the gas based on the representation of the sensor voltage.

5. The device of description 4, wherein the substance is ethanol and the property is a concentration of ethanol in the gas.

6. The device of description 5, wherein the processor is further configured to generate an indication of blood alcohol content of a person that exhaled the gas based on the concentration of the ethanol in the gas.

7. The device of either description 4 or 5, wherein the substance is ethanol and the property is a blood alcohol content of a person that exhaled the gas.

8. The device of any of descriptions 4-7, wherein the substance is carbon monoxide and the property is a concentration of carbon monoxide in the gas.

9. The device of any of descriptions 48, further comprising: a second electronic sensor configured to emit a second sensor voltage used on water vapor detected in a gas passed over the second sensor; and wherein the processor is configured to determine the property of the gas based on the second sensor voltage.

10. The device of description 9, further comprising:
a third electronic sensor configured to emit a third sensor voltage based on is temperature of the gas; and wherein
the processor is configured to determine the property based on the third sensor voltage.

11. The device of any of descriptions 1-10, further comprising:
a flow-control tube that guides the gas over the sensor.

12. A system comprising:
a property-detection device comprising:
   an electronic gas sensor configured to generate a sensor voltage based on an amount of a substance detected in a gas to which the sensor is exposed;
   an audio jack, communicatively coupled to the electronic gas sensor, configured to output a representation of the sensor voltage via a communications protocol;
a processor configured to:
   receive the representation of the sensor voltage outputted from the audio jack; and
   determine a property of the gas based on representation of the sensor voltage; and
   send the property of the gas and a location of the device over a network; and
a property-processing device comprising:
   a processor configured to:
      receive the property of the gas and the location of the device;
      store the property and the location of the device in a database;
      transmit a request based on the property and the location of the device.

13. The system of description 12, wherein:
the substance is ethanol,
the property is a concentration of ethanol in the gas, and
the request is for a taxi cab to be sent to the location of the device.

14. The system of description 12, wherein:
the substance is a pathogen,
the property is a concentration of the pathogen in the gas, and
the request is for a medical service to acknowledge a health risk at the location of the device.

15. A method comprising:
receiving a representation of a sensor voltage output from an audio jack, the output generated by an electronic gas sensor in response to exposure to a gas exhaled by an
based, on the representation of the sensor voltage, determining; the blood-alcohol content of the individual;
causing the blood-alcohol content of the individual to be displayed.

16. The method of description 15, wherein identifying the blood-alcohol content of the individual accesses a table in a memory, the table storing a plurality of values corresponding to a plurality of sensor voltages.

17. A method comprising;
receiving a representation of a sensor voltage output from an audio jack, the output generated by an electronic gas sensor;
based on thee representation of the sensor voltage, identifying a property of the gas; and
causing the property of the gas to be displayed.

18. The method of description 17, further comprising:
sending the property of the gas over a network; and
receiving a recommendation based on the property.

19. The method of description 18, wherein;
the property is the ethanol concentration of the gas, and
the recommendation includes contacting a taxi company.

20. The method of description 19, further comprising:
generating an indication of blood alcohol content of a person that exhaled the gas based on the concentration of the ethanol in the gas.

What is claimed is:

1. A device comprising:
a housing,
an intake port through which a gas can pass into the housing;
an electronic gas sensor disposed within the housing, the electronic gas sensor configured to generate a sensor voltage based on an amount of a substance detected in the gas;
a processor configured to convert the sensor voltage to a result value; and
an audio jack protruding from the housing, the audio jack communicatively coupled to the electronic gas sensor and configured to output the result value via a communications protocol.

2. The device of claim 1, wherein
the result value is a representation of the sensor voltage.

3. The device of claim 1, wherein
the result value represents a property of the gas.

4. The device of claim 1, further comprising:
a processor configured to:
receive the representation of the sensor voltage outputted from the audio jack; and
determine a property of the gas based on the representation of the sensor voltage.

5. The device of claim 4, wherein the substance is ethanol and the property is a concentration of ethanol in the gas.

6. The device of claim 5, wherein the processor is further configured to generate an indication of blood alcohol content of a person that exhaled the gas based on the concentration of the ethanol in the gas.

7. The device of claim 4, wherein the substance is ethanol and the property is a blood alcohol content of a person that exhaled the gas.

8. The device of claim 4, wherein the substance is carbon monoxide and the property is a concentration of carbon monoxide in the gas.

9. The device of claim 4, further comprising:
a second electronic sensor configured to emit a second sensor voltage based on water vapor detected in a gas passed over the second sensor; and wherein
the processor is configured to determine the property of the gas based on the second sensor voltage.

10. The device of claim 9, further comprising:
a third electronic sensor configured to emit a third sensor voltage based on a temperature of the gas; and wherein
the processor is configured to determine the property based on the third sensor voltage,

11. The device of claim 1, further comprising:
as flow-control tube that guides the gas over the sensor.

12. A system comprising:
a property-detection device comprising:
an electronic gas sensor configured to generate a sensor voltage based on an amount of a substance detected in a gas to which the sensor is exposed;
an audio jack, communicatively coupled to the electronic gas sensor, configured to output a representation of the sensor voltage via a communications protocol;
a processor configured to:
receive the representation of the sensor voltage outputted from the audio jack; and
determine a property of the as based on representation of the sensor voltage; and
send the property of the gas and a location of the device over a network; and
a property-processing device comprising:
a processor configured to:
receive the property of the gas and the location of the device;
store the property and the location of the device in a database:,
transmit a request based on the property and the location of the device.

13. The system of claim 12, wherein:
the substance is ethanol,
the property is a concentration of ethanol in the gas, and
request is for a taxi cab to be sent to the location of the device.

14. The system of claim 12, wherein:
the substance is a pathogen,
the property is a concentration of the pathogen in the gas, and
the request is for a medical service to acknowledge a health risk at the location of the device.

15. A method comprising:
receiving a representation of a sensor voltage output from an audio jack, the output generated by an electronic gas sensor in response to exposure to a gas exhaled by an individual;
based on the representation of the sensor voltage, determining the blood-alcohol content of the individual;
causing the blood-alcohol content of the individual to be displayed.

16. The method of claim 15, wherein identifying the blood-alcohol content of the individual accesses a table in a memory, the table storing a plurality of values corresponding to a plurality of sensor voltages.

17. A method comprising:
receiving a representation of a sensor voltage output from an audio jack, the output generated by an electronic gas sensor;
based on the representation of the sensor voltage, identifying a property of the gas; and
causing the property of the gas to be displayed.

18. The method of claim 17, further comprising:
sending the property of the gas over a network; and
receiving a recommendation based on the property.

19. The method of claim 18, wherein:
the property is the ethanol concentration of the gas, and
the recommendation includes contacting taxi company.

20. The method of claim 19, farther comprising:
generating an indication of blood alcohol content of a person that exhaled the gas based on the concentration of the ethanol in the gas.
